(19) United States
(12) Patent Application Publication
(10) Pub. No.: US 2011/0038801 A1
(43) Pub. Date: Feb. 17, 2011

(54) METHODS AND COMPOSITIONS FOR IDENTIFYING LUNG CANCER OR A HUMORAL IMMUNE RESPONSE AGAINST LUNG CANCER

(75) Inventors: Karin Jooss, San Diego, CA (US); Andrew D. Simmons, San Mateo, CA (US)

Correspondence Address: JONES DAY, 222 EAST 41ST ST, NEW YORK, NY, 10017, US

(73) Assignee: BioSante Pharmaceuticals, Inc., Lincolnshire, IL (US)

(21) Appl. No.: 12/810,002

(22) PCT Filed: Dec. 19, 2008
(86) PCT No.: PCT/US08/13948

§ 371 Date: Nov. 1, 2010

Related U.S. Application Data
(60) Provisional application No. 61/008,847, filed on Dec. 21, 2007.

(51) Int. Cl. A61B 5/055; A61K 49/00; C40B 30/04; G01N 33/53; C40B 60/12; A61P 35/00; A61P 37/00; G06F 19/00

(52) U.S. Cl. 424/9.3; 424/9.1; 506/9; 435/7.24; 506/39; 702/19

(57) ABSTRACT

Methods and compositions are provided for identifying lung cancer or a humoral immune response against lung cancer. Also disclosed are methods for determining whether a subject is responding or is likely to respond to lung cancer immunotherapy.

### 1. FIELD

Provided herein are lung cancer markers, compositions comprising such markers, immunoglobulins specific for such markers, and methods of using such markers and/or immunoglobulins to assess an immune response against lung cancer. An immune response against the markers correlates with an immune response, in particular a humoral immune response, against cancer cells which immune response is preferably associated with prophylaxis of lung cancer, treatment of lung cancer, and/or amelioration of at least one symptom associated with lung cancer.

### 2. BACKGROUND

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for cancer including chemotherapy, surgery, radiation therapy and cellular therapy have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity.

One therapeutic approach to cancer treatment involves the use of genetically modified tumor cells which express cytokines locally at the immunotherapy site. Activity has been demonstrated in tumor models using a variety of immunomodulatory cytokines, including IL-4, IL-2, TNF-alpha, G-CSF, IL-7, IL-6 and GM-CSF, as described in Golumbeck P T et al., Science 254:13-716, 1991; Gansbacher B et al., J. Exp. Med. 172:1217-1224, 1990; Fearon E R et al., Cell 60:397-403, 1990; Gansbacher B et al., Cancer Res. 50:7820-25, 1990; Teng M et al., PNAS 88:3535-3539, 1991; Columbo M P et al., J. Exp. Med. 174:1291-1298, 1991; Aoki et al., Proc Natl Acad Sci USA. 89(9):3850-4, 1992; Porgador A, et al., Nat. Immun. 13(2-3):113-30, 1994; DranoffG et al., PNAS 90:3539-3543, 1993; Lee C T et al., Human Gene Therapy 8:187-193, 1997; Nagai E et al., Cancer Immunol. Immunother. 47:2-80, 1998 and Chang A et al., Human Gene Therapy 11:839-850, 2000, respectively. The use of autologous cancer cells as immunotherapy to augment anti-tumor immunity has been explored for some time. See, e.g., Oettgen et al., “The History of Cancer Immunotherapy,” In: Biologic Therapy of Cancer, Devita et al. (eds.) J. Lippincot Co., pp 87-199, 1991; Armstrong T D and Jaffee E M, Surg Oncol Clin N Am. 11(3):681-96, 2002; and Bodey B et al., Anticancer Res 20(4):2665-76, 2000).

Several phase I/II human trials using GM-CSF-secreting autologous or allogeneic tumor cell vaccines have been performed (Simons et al. Cancer Res 1999 59:5160-8; Soiffer et al. Proc Natl Acad Sci USA 1998 95:13141-6; Simons et al. Cancer Res 1997 57:1537-46; Jaffee et al. J Clin Oncol 2001 19:145-56; Salgia et al. J Clin Oncol 2003 21:624-30; Soiffer et al. J Clin Oncol 2003 21:3343-50; Nemunaitis et al. J Natl Cancer Inst. 2004 Feb. 18 96(4):326-31; Borello and Pardoll, Growth Factor Rev. 13(2):185-93, 2002; and Thomas et al., J. Exp. Med. 200(3)297-306, 2004).

Administration of genetically modified GM-CSF-expressing cancer cells to a patient results in an immune response, and preliminary clinical efficacy against lung and other cancers has been demonstrated in Phase I/II clinical trails. However, there remains a need for improved methods and compositions for predicting whether such therapies are likely to be effective, for monitoring the effectiveness of such therapies, and for increasing the effectiveness of such therapies. These and other needs are provided by the methods and compositions provided herein.

### 3. SUMMARY

Provided herein are lung cancer markers, compositions comprising such markers, immunoglobulins specific for such markers, and methods of using such markers and/or immunoglobulins to assess an immune response against lung cancer. An immune response against the markers correlates with an immune response, in particular a humoral immune response, against cancer cells which immune response is preferably associated with prophylaxis of lung cancer, treatment of lung cancer, and/or amelioration of at least one symptom associated with lung cancer. In certain embodiments, the lung cancer is non-small cell lung cancer (NSCLC).

Thus, in a first aspect, provided herein is a method for identifying whether a subject is afflicted with lung cancer, comprising detecting an immune response against an antigen identified in Table 2, 3 or 4, wherein detection of the immune response indicates that the subject is afflicted with lung cancer. In certain embodiments, an immune response is detected against an antigen identified in Table 2. In certain embodiments, an immune response is detected against an antigen identified in Table 3. In certain embodiments, an immune response is detected against an antigen identified in Table 4. In certain embodiments, an immune response is detected against 1, 2, 3, 4, 5, 6, 7, 8, 9, 10, 11, 12, 13, 14, 15 or more of the antigens in Table 2. In certain embodiments, an immune response is detected against 1, 2, 3, 4, 5, 6, 7, 8, 9, 10, 11, 12, 13, 14, 15 or more of the antigens in Table 3. In certain embodiments, an immune response is detected against 1, 2, 3, 4, 5, 6, 7, 8, 9, 10, 11, 12, 13, 14, 15 or more of the antigens in Table 4. In certain embodiments, the lung cancer is non-small cell lung cancer (NSCLC).

In certain embodiments, the subject is a mammal. In certain embodiments, the subject is a human. In certain embodiments, the immune response is a humoral immune response. In certain embodiments, the immune response is a cellular immune response.

In another aspect, provided herein is a method for determining whether a subject is likely to respond to lung cancer therapy with a composition comprising cancer cells that have been rendered proliferation-incompetent and have been genetically engineered to express GM-CSF, comprising detecting an immune response against an antigen listed in Table 2, 3 or 4, wherein detecting the immune response indicates that the subject is likely to respond to said lung cancer therapy. In certain embodiments, the lung cancer therapy is for the treatment of non-small cell lung cancer (NSCLC). In certain embodiments, the lung cancer therapy can be other than a therapy with a composition comprising cancer cells that have been rendered proliferation-incompetent and have been genetically engineered to express GM-CSF; in such embodiments, the lung cancer therapy can be any cancer immunotherapy known to one skilled in the art without limitation.

In certain embodiments, the subject is a mammal. In certain embodiments, the subject is a human. In certain embodiments, the cancer cells are autologous. In certain embodiments, the cancer cells are allogeneic. In certain embodiments, the cancer cells are LnCaP cells or PC3 cells. In some embodiments, the cancer cells are NCIH838 cells, NCIH1623 cells or NCIH1435 cells.

In certain embodiments, an immune response is detected against an antigen identified in Table 2. In certain embodiments, an immune response is detected against an antigen identified in Table 3. In certain embodiments, an immune response is detected against an antigen identified in Table 4. In certain embodiments, an immune response is detected against 1, 2, 3, 4, 5, 6, 7, 8, 9, 10, 11, 12, 13, 14, 15 or more of the antigens in Table 2. In certain embodiments, an immune response is detected against 1, 2, 3, 4, 5, 6, 7, 8, 9, 10, 11, 12, 13, 14, 15 or more of the antigens in Table 3. In certain embodiments, an immune response is detected against 1, 2, 3, 4, 5, 6, 7, 8, 9, 10, 11, 12, 13, 14, 15 or more of the antigens in Table 4.

In certain embodiments, responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, increased impact on circulating T cell and dendritic cell numbers, phenotype, and/or function, cytokine response, reduced metastasis as measured by bone scan/MRI or other methods, increased time to progression, decreased serum concentrations of ICTP, decreased concentrations of serum C-reactive protein or decreased numbers of circulating tumor cells (CTCs).

In certain embodiments, responsiveness to the cancer therapy is measured by decreased serum concentrations of tumor specific markers. In certain embodiments, responsiveness to the cancer therapy is measured by increased overall survival time. In certain embodiments, responsiveness to the cancer therapy is measured by increased progression-free survival. In certain embodiments, responsiveness to the cancer therapy is measured by decreased tumor size. In certain embodiments, responsiveness to the cancer therapy is measured by decreased metastasis marker response. In certain embodiments, responsiveness to the cancer therapy is measured by increased impact on minimal residual disease. In certain embodiments, responsiveness to the cancer therapy is measured by increased induction of antibody response to the cancer cells that have been rendered proliferation-incompetent. In certain embodiments, responsiveness to the cancer therapy is measured by increased induction of delayed-type-hypersensitivity (DTH) response to injections of autologous tumor. In certain embodiments, responsiveness to the cancer therapy is measured by increased induction of T cell response to autologous tumor or candidate tumor-associated antigens or decreased numbers of circulating tumor cells (CTCs). In certain embodiments, responsiveness to the cancer therapy is measured by increased impact on circulating T cell and dendritic cell numbers, phenotype, and/or function. In certain embodiments, responsiveness to the cancer therapy is measured by cytokine response. In certain embodiments, responsiveness to the cancer therapy is measured by decreased numbers of circulating tumor cells (CTCs).

In certain embodiments, the immune response is a humoral immune response. In certain embodiments, the immune response is a cellular immune response.

In another aspect, provided herein is a computer-implemented method for determining whether a subject is likely to respond to lung cancer therapy with a composition comprising cancer cells that have been rendered proliferation-incompetent and have been genetically engineered to express GM-CSF, comprising inputting into a computer memory data indicating whether an immune response against an antigen listed in Table 2, 3 or 4 is detected, inputting into the computer memory a correlation between an immune response against an antigen listed in Table 2, 3, or 4 and a likelihood of responding to said therapy, and determining whether the subject is likely to respond to said therapy. In certain embodiments, an immune response is detected against an antigen identified in Table 2. In certain embodiments, an immune response is detected against an antigen identified in Table 3. In certain embodiments, an immune response is detected against an antigen identified in Table 4. In certain embodiments, an immune response is detected against 1, 2, 3, 4, 5, 6, 7, 8, 9, 10, 11, 12, 13, 14, 15 or more of the antigens in Table 2. In certain embodiments, an immune response is, detected against 1, 2, 3, 4, 5, 6, 7, 8, 9, 10, 11, 12, 13, 14, 15 or more of the antigens in Table 3. In certain embodiments, an immune response is detected against 1, 2, 3, 4, 5, 6, 7, 8, 9, 10, 11, 12, 13, 14, 15 or more of the antigens in Table 4. In certain embodiments, the lung cancer therapy is for the treatment of non-small cell lung cancer (NSCLC).

In certain embodiments, the subject is a mammal. In certain embodiments, the subject is a human.

In certain embodiments, the cancer cells are autologous. In certain embodiments, the cancer cells are allogeneic. In certain embodiments, the cancer cells are LnCaP cells or PC3 cells. In some embodiments, the cancer cells are NCIH838 cells, NCIH1623 cells or NCIH1435 cells.

In certain embodiments, responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, increased impact on circulating T cell and dendritic cell numbers, phenotype, and/or function, cytokine response, reduced metastasis as measured by bone scan/MRI, increased time to progression, decreased serum concentrations of ICTP, or decreased concentrations of serum C-reactive protein or decreased numbers of circulating tumor cells. In certain embodiments, responsiveness to the cancer therapy is measured by decreased numbers of circulating tumor cells.

In certain embodiments, responsiveness to the cancer therapy is measured by decreased serum concentrations of tumor specific markers. In certain embodiments, responsiveness to the cancer therapy is measured by increased overall survival time. In certain embodiments, responsiveness to the cancer therapy is measured by increased progression-free survival. In certain embodiments, responsiveness to the cancer therapy is measured by decreased tumor size. In certain embodiments, responsiveness to the cancer therapy is measured by decreased metastasis marker response. In certain embodiments, responsiveness to the cancer therapy is measured by increased impact on minimal residual disease. In certain embodiments, responsiveness to the cancer therapy is measured by increased induction of antibody response to the cancer cells that have been rendered proliferation-incompetent. In certain embodiments, responsiveness to the cancer therapy is measured by increased induction of delayed-type-hypersensitivity (DTH) response to injections of autologous tumor. In certain embodiments, responsiveness to the cancer therapy is measured by increased induction of T cell response to autologous tumor or candidate tumor-associated antigens. In certain embodiments, responsiveness to the cancer therapy is measured by increased impact on circulating T cell and dendritic cell numbers, phenotype, and/or function. In certain embodiments, responsiveness to the cancer therapy is measured by cytokine response. In certain embodiments, responsiveness to the cancer therapy is measured by decreased numbers of circulating tumor cells.

In certain embodiments, the immune response is a humoral immune response. In certain embodiments, the immune response is a cellular immune response.

In another aspect, provided herein is a method for determining whether a subject is responding to lung cancer therapy with a composition comprising cancer cells that have been rendered proliferation-incompetent and have been genetically engineered to express GM-CSF, comprising administering an effective amount of a composition comprising cancer cells that have been rendered proliferation-incompetent and have been genetically engineered to express GM-CSF, and detecting an immune response against an antigen listed in Table 2, 3 or 4, wherein detecting the immune response indicates that the subject is responding to said lung cancer therapy. In certain embodiments, an immune response is detected against an antigen identified in Table 2. In certain embodiments, an immune response is detected against an antigen identified in Table 3. In certain embodiments, an immune response is detected against an antigen identified in Table 4. In certain embodiments, an immune response is detected against 1, 2, 3, 4, 5, 6, 7, 8, 9, 10, 11, 12, 13, 14, 15 or more of the antigens in Table 2. In certain embodiments, an immune response is detected against 1, 2, 3, 4, 5, 6, 7, 8, 9, 10, 11, 12, 13, 14, 15 or more of the antigens in Table 3. In certain embodiments, an immune response is detected against 1, 2, 3, 4, 5, 6, 7, 8, 9, 10, 11, 12, 13, 14, 15 or more of the antigens in Table 4. In certain embodiments, the lung cancer therapy is for the treatment of non-small cell lung cancer (NSCLC).

In certain embodiments, the subject is a mammal. In certain embodiments, the subject is a human. In certain embodiments, the cancer cells are autologous. In certain embodiments, the cancer cells are allogeneic. In certain embodiments, the cancer cells are LnCaP cells or PC3 cells. In some embodiments, the cancer cells are NCIH838 cells, NCIH1623 cells or NCIH1435 cells.

In certain embodiments, responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, or increased impact on circulating T cell and dendritic cell numbers, phenotype, and/or function, cytokine response, or decreased numbers of circulating tumor cells.

In certain embodiments, responsiveness to the cancer therapy is measured by decreased serum concentrations of tumor specific markers. In certain embodiments, responsiveness to the cancer therapy is measured by increased overall survival time. In certain embodiments, responsiveness to the cancer therapy is measured by increased progression-free survival. In certain embodiments, responsiveness to the cancer therapy is measured by decreased tumor size. In certain embodiments, responsiveness to the cancer therapy is measured by decreased metastasis marker response. In certain embodiments, responsiveness to the cancer therapy is measured by increased impact on minimal residual disease. In certain embodiments, responsiveness to the cancer therapy is measured by increased induction of antibody response to the cancer cells that have been rendered proliferation-incompetent. In certain embodiments, responsiveness to the cancer therapy is measured by increased induction of delayed-type-hypersensitivity (DTH) response to injections of autologous tumor. In certain embodiments, responsiveness to the cancer therapy is measured by increased induction of T cell response to autologous tumor or candidate tumor-associated antigens. In certain embodiments, wherein responsiveness to the cancer therapy is measured by increased impact on circulating T cell and dendritic cell numbers, phenotype, and/or function, cytokine response, or decreased numbers of circulating tumor cells.

In certain embodiments, the immune response is a humoral immune response. In certain embodiments, the immune response is a cellular immune response.

In yet another aspect, provided herein is a computer-implemented method for determining whether a subject responding to lung cancer therapy with a composition comprising cancer cells that have been rendered proliferation-incompetent and have been genetically engineered to express GM-CSF, comprising administering an effective amount of a composition comprising cancer cells that have been rendered proliferation-incompetent and have been genetically engineered to express GM-CSF, inputting into a computer memory data indicating whether an immune response against an antigen listed in Table 2, 3 or 4 is detected, inputting into the computer memory a correlation between an immune response against an antigen listed in Table 2, 3 or 4 and responsiveness to said therapy, and determining whether the subject is responding to said therapy. In certain embodiments, an immune response is detected against an antigen identified in Table 2. In certain embodiments, an immune response is detected against an antigen identified in Table 3. In certain embodiments, an immune response is detected against an antigen identified in Table 4. In certain embodiments, an immune response is detected against 1, 2, 3, 4, 5, 6, 7, 8, 9, 10, 11, 12, 13, 14, 15 or more of the antigens in Table 2. In certain embodiments, an immune response is detected against 1, 2, 3, 4, 5, 6, 7, 8, 9, 10, 11, 12, 13, 14, 15 or more of the antigens in Table 3. In certain embodiments, an immune response is detected against 1, 2, 3, 4, 5, 6, 7, 8, 9, 10, 11, 12, 13, 14, 15 or more of the antigens in Table 4. In certain embodiments, the lung cancer therapy is for the treatment of non-small cell lung cancer (NSCLC).

In certain embodiments, the subject is a mammal. In certain embodiments, the subject is a human.

In certain embodiments, the cancer cells are autologous. In certain embodiments, the cancer cells are allogeneic. In certain embodiments, the cancer cells are LnCaP cells or PC3 cells. In some embodiments, the cancer cells are NCIH838 cells, NCIH1623 cells or NCIH1435 cells.

In certain embodiments, responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, or increased impact on circulating T cell and dendritic cell numbers, phenotype, and/or function, cytokine response, or decreased numbers of circulating tumor cells.

In certain embodiments, responsiveness to the cancer therapy is measured by decreased serum concentrations of tumor specific markers. In certain embodiments, responsiveness to the cancer therapy is measured by increased overall survival time. In certain embodiments, responsiveness to the cancer therapy is measured by increased progression-free survival. In certain embodiments, responsiveness to the cancer therapy is measured by decreased tumor size. In certain embodiments, responsiveness to the cancer therapy is measured by decreased metastasis marker response. In certain embodiments, responsiveness to the cancer therapy is measured by increased impact on minimal residual disease. In certain embodiments, responsiveness to the cancer therapy is measured by increased induction of antibody response to the cancer cells that have been rendered proliferation-incompetent. In certain embodiments, responsiveness to the cancer therapy is measured by increased induction of delayed-type-hypersensitivity (DTH) response to injections of autologous tumor. In certain embodiments, responsiveness to the cancer therapy is measured by increased induction of T cell response to autologous tumor or candidate tumor-associated antigens. In certain embodiments, responsiveness to the cancer therapy is measured by increased impact on circulating T cell and dendritic cell numbers, phenotype, and/or function, cytokine response, or decreased numbers of circulating tumor cells.

In certain embodiments, the immune response is a humoral immune response. In certain embodiments, the immune response is a cellular immune response.

In yet another aspect, provided herein is a method for determining whether a subject is responding to lung cancer therapy with a composition comprising cancer cells that have been rendered proliferation-incompetent and have been genetically engineered to express GM-CSF, comprising detecting an immune response against an antigen listed in Table 2, 3 or 4 at a first time, administering an effective amount of a composition comprising cancer cells that have been rendered proliferation-incompetent and have been genetically engineered to express GM-CSF, and detecting an immune response against the antigen listed in Table 2, 3 or 4 at a later second time, wherein an increase in the immune response detected at the later second time relative to the earlier first time indicates that the subject is responding to said lung cancer therapy. In certain embodiments, an immune response is detected at the first and second times against an antigen identified in Table 2. In certain embodiments, an immune response is detected at the first and second times against an antigen identified in Table 3. In certain embodiments, an immune response is detected at the first and second times against an antigen identified in Table 4. In certain embodiments, an immune response is detected at the first and second times against 1, 2, 3, 4, 5, 6, 7, 8, 9, 10, 11, 12, 13, 14, 15 or more of the antigens in Table 2. In certain embodiments, an immune response is detected at the first and second times against 1, 2, 3, 4, 5, 6, 7, 8, 9, 10, 11, 12, 13, 14, 15 or more of the antigens in Table 3. In certain embodiments, an immune response is detected at the first and second times against 1, 2, 3, 4, 5, 6, 7, 8, 9, 10, 11, 12, 13, 14, 15 or more of the antigens in Table 4. In certain embodiments, the lung cancer therapy is for the treatment of non-small cell lung cancer (NSCLC).

In certain embodiments, the subject is a mammal. In certain embodiments, wherein the subject is a human. In certain embodiments, the cancer cells are autologous. In certain embodiments, the cancer cells are allogeneic. In certain embodiments, the cancer cells are LnCaP cells or PC3 cells. In some embodiments, the cancer cells are NCIH838 cells, NCIH1623 cells or NCIH1435 cells. In certain embodiments, responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, increased impact on circulating T cell and dendritic cell numbers, phenotype, and/or function, cytokine response, reduced metastasis as measured by bone scan/MRI or other methods, increased time to progression, decreased serum concentrations of ICTP, decreased concentrations of serum C-reactive protein or decreased numbers of circulating tumor cells (CTCs).

In certain embodiments, responsiveness to the cancer therapy is measured by decreased serum concentrations of tumor specific markers. In certain embodiments, responsiveness to the cancer therapy is measured by increased overall survival time. In certain embodiments, responsiveness to the cancer therapy is measured by increased progression-free survival. In certain embodiments, responsiveness to the cancer therapy is measured by decreased tumor size. In certain embodiments, responsiveness to the cancer therapy is measured by decreased metastasis marker response. In certain embodiments, responsiveness to the cancer therapy is measured by increased impact on minimal residual disease. In certain embodiments, responsiveness to the cancer therapy is measured by increased induction of antibody response to the cancer cells that have been rendered proliferation-incompetent. In certain embodiments, responsiveness to the cancer therapy is measured by increased induction of delayed-type-hypersensitivity (DTH) response to injections of autologous tumor. In certain embodiments, responsiveness to the cancer therapy is measured by increased induction of T cell response to autologous tumor or candidate tumor-associated antigens. In certain embodiments, responsiveness to the cancer therapy is measured by increased impact on circulating T cell and dendritic cell numbers, phenotype, and/or function. In certain embodiments, responsiveness to the cancer therapy is measured by cytokine response. In certain embodiments, responsiveness to the cancer therapy is measured by reduced metastasis as measured by bone scan/MRI or other methods. In certain embodiments, responsiveness to the cancer therapy is measured by increased time to progression. In certain embodiments, responsiveness to the cancer therapy is measured by decreased serum concentrations of ICTP. In certain embodiments, responsiveness to the cancer therapy is measured by decreased concentrations of serum C-reactive protein. In certain embodiments, responsiveness to the cancer therapy is measured by decreased numbers of circulating tumor cells.

In certain embodiments, the immune response detected at the first and second times is a humoral immune response. In certain embodiments, the immune response detected at the first and second times is a cellular immune response.

In still another aspect, provided herein is a computer-implemented method for determining whether a subject is responding to lung cancer therapy with a composition comprising cancer cells that have been rendered proliferation-incompetent and have been genetically engineered to express GM-CSF, comprising administering an effective amount of a composition comprising cancer cells that have been rendered proliferation-incompetent and have been genetically engineered to express GM-CSF, inputting into a computer memory data indicating whether an immune response against an antigen listed in Table 2, 3 or 4 is detected at a first time prior to said step of administering and at a later second time subsequent to said step of administering, inputting into the computer memory a correlation between an increase in the immune response against the antigen listed in Table 2, 3 or 4 at said later second time relative to said earlier first time and responsiveness to said therapy, and determining whether the subject is responding to said therapy.

In certain embodiments, an immune response is detected at the first and second times against an antigen identified in Table 2. In certain embodiments, an immune response is detected at the first and second times against an antigen identified in Table 3. In certain embodiments, an immune response is detected at the first and second times against an antigen identified in Table 4. In certain embodiments, an immune response is detected at the first and second times against 1, 2, 3, 4, 5, 6, 7, 8, 9, 10, 11, 12, 13, 14, 15 or more of the antigens in Table 2. In certain embodiments, an immune response is detected at the first and second times against 1, 2, 3, 4, 5, 6, 7, 8, 9, 10, 11, 12, 13, 14, 15 or more of the antigens in Table 3. In certain embodiments, an immune response is detected at the first and second times against 1, 2, 3, 4, 5, 6, 7, 8, 9, 10, 11, 12, 13, 14, 15 or more of the antigens in Table 4. In certain embodiments, the lung cancer therapy is for the treatment of non-small cell lung cancer (NSCLC).

In certain embodiments, the subject is a mammal. In certain embodiments, the subject is a human. In certain embodiments, the cancer cells are autologous. In certain embodiments, the cancer cells are allogeneic. In certain embodiments, the cancer cells are LnCaP cells or PC3 cells. In some embodiments, the cancer cells are NCIH838 cells, NCIH1623 cells or NCIH1435 cells.

In certain embodiments, responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, increased impact on circulating T cell and dendritic cell numbers, phenotype, and function, cytokine response, reduced metastasis as measured by bone scan/MRI, increased time to progression, decreased serum concentrations of ICTP, decreased concentrations of serum C-reactive protein or decreased numbers of circulating tumor cells.

In certain embodiments, responsiveness to the cancer therapy is measured by decreased serum concentrations of tumor specific markers. In certain embodiments, responsiveness to the cancer therapy is measured by increased overall survival time. In certain embodiments, responsiveness to the cancer therapy is measured by increased progression-free survival. In certain embodiments, responsiveness to the cancer therapy is measured by decreased tumor size. In certain embodiments, responsiveness to the cancer therapy is measured by decreased metastasis marker response. In certain embodiments, responsiveness to the cancer therapy is measured by increased impact on minimal residual disease. In certain embodiments, responsiveness to the cancer therapy is measured by increased induction of antibody response to the cancer cells that have been rendered proliferation-incompetent. In certain embodiments, responsiveness to the cancer therapy is measured by increased induction of delayed-type-hypersensitivity (DTH) response to injections of autologous tumor. In certain embodiments, responsiveness to the cancer therapy is measured by increased induction of T cell response to autologous tumor or candidate tumor-associated antigens. In certain embodiments, responsiveness to the cancer therapy is measured by increased impact on circulating T cell and dendritic cell numbers, phenotype, and/or function. In certain embodiments, responsiveness to the cancer therapy is measured by cytokine response. In certain embodiments, responsiveness to the cancer therapy is measured by reduced metastasis as measured by bone scan/MRI. In certain embodiments, responsiveness to the cancer therapy is measured by increased time to progression. In certain embodiments, responsiveness to the cancer therapy is measured by decreased serum concentrations of ICTP. In certain embodiments, responsiveness to the cancer therapy is measured by decreased concentrations of serum C-reactive protein. In certain embodiments, responsiveness to the cancer therapy is measured by decreased numbers of circulating tumor cells.

In certain embodiments, the immune response is a humoral immune response. In certain embodiments, the immune response is a cellular immune response.

In still another aspect, provided herein is a computer-readable media embedded with computer executable instructions for performing a method described herein.

In yet another aspect, provided herein is a computer system configured to perform a method described herein.

### 4. DETAILED DESCRIPTION

Provided herein are lung cancer markers, compositions comprising such markers, immunoglobulins specific for such markers, and methods of using such markers and/or immunoglobulins to assess an immune response against cancer. The markers, compositions, immunoglobulins, and methods are useful, for example, for assessing an immune response, in particular a humoral immune response, against cancer cells which immune response is preferably associated with prophylaxis of lung cancer, treatment of lung cancer, and/or amelioration of at least one symptom associated with lung cancer. In certain embodiments, the lung cancer is non-small cell lung cancer (NSCLC).

Without intending to be bound to any particular theory or mechanism of action, it is believed that one aspect of the immune response induced by therapy with genetically modified tumor cells that express a cytokine is an immune response against certain polypeptides expressed by the genetically modified tumor cell and/or cells from the tumor afflicting the subject. It is also believed that this immune response plays an important role in the effectiveness of this therapy to treat, e.g., non-small cell lung cancer.

### 4.1 DEFINITIONS

By the term “cytokine” or grammatical equivalents, herein is meant the general class of hormones of the cells of the immune system, including lymphokines, monokines, and others. The definition includes, without limitation, those hormones that act locally and do not circulate in the blood, and which, when used in accord with the methods provided herein, will result in an alteration of an individual's immune response. The term “cytokine” or “cytokines” as used herein refers to the general class of biological molecules, which affect cells of the immune system. The definition is meant to include, but is not limited to, those biological molecules that act locally or may circulate in the blood, and which, when used in the compositions or methods provided herein, serve to regulate or modulate an individual's immune response to cancer. Exemplary cytokines for use in practicing the methods provided herein include, but are not limited to, interferon-alpha (IFN-alpha), IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, ICAM, macrophage colony stimulating factor (M-CSF), granulocyte colony stimulating factor (G-CSF) and granulocyte-macrophage colony stimulating factor (GM-CSF).

As used herein, the terms “cancer”, “cancer cells”, “neoplastic cells”, “neoplasia”, “tumor”, and “tumor cells” (used interchangeably) refer to cells that exhibit relatively autonomous growth, so that they exhibit an aberrant growth phenotype or aberrant cell status characterized by a significant loss of control of cell proliferation. A tumor cell may be a hyperplastic cell, a cell that shows a lack of contact inhibition of growth in vitro or in vivo, a cell that is incapable of metastasis in vivo, or a cell that is capable of metastasis in vivo. Neoplastic cells can be malignant or benign. It follows that cancer cells are considered to have an aberrant cell status. “Tumor cells” may be derived from a primary tumor or derived from a tumor metastases. The “tumor cells” may be recently isolated from a patient (a “primary tumor cell”) or may be the product of long term in vitro culture.

The term “primary tumor cell” is used in accordance with the meaning in the art. A primary tumor cell is a cancer cell that is isolated from a tumor in a mammal and has not been extensively cultured in vitro.

The term “antigen from a tumor cell” and “tumor antigen” and “tumor cell antigen” may be used interchangeably herein and refer to any protein, peptide, carbohydrate or other component derived from or expressed by a tumor cell which is capable of eliciting an immune response. The definition is meant to include, but is not limited to, whole tumor cells, tumor cell fragments, plasma membranes taken from a tumor cell, proteins purified from the cell surface or membrane of a tumor cell, unique carbohydrate moieties associated with the cell surface of a tumor cell or tumor antigens expressed from a vector in a cell. The definition also includes those antigens from the surface of the cell, which require special treatment of the cells to access.

The term “genetically modified tumor cell” as used herein refers to a composition comprising a population of cells that has been genetically modified to express a transgene, and that is administered to a patient as part of a cancer treatment regimen. The genetically modified tumor cell immunotherapy comprises tumor cells which are “autologous” or “allogeneic” to the patient undergoing treatment or “bystander cells” that are mixed with tumor cells taken from the patient. A GM-CSF-expressing genetically modified tumor cell immunotherapy may be referred to herein as “GVAX”®. Autologous and allogeneic cancer cells that have been genetically modified to express a cytokine, e.g., GM-CSF, followed by readministration to a patient for the treatment of cancer are described in U.S. Pat. Nos. 5,637,483, 5,904,920, 6,277,368 and 6,350,445, each of which is expressly incorporated by reference herein. A form of GM-CSF-expressing genetically modified cancer cells or a “cytokine-expressing cellular immunotherapy” for the treatment of pancreatic cancer is described in U.S. Pat. Nos. 6,033,674 and 5,985,290, both of which are expressly incorporated by reference herein. A universal immunomodulatory cytokine-expressing bystander cell line is described in U.S. Pat. No. 6,464,973, expressly incorporated by reference herein.

The term “enhanced expression” as used herein, refers to a cell producing higher levels of a particular protein than would be produced by the naturally occurring cell or the parental cell from which it was derived. Cells may be genetically modified to increase the expression of a cytokine, such as GM-CSF, or an antigen that elicits an immune response following administration of a cytokine-expressing cellular immunotherapy, such as GVAX®. The expression of an endogenous antigen may be increased using any method known in the art, such as genetically modifying promoter regions of genomic sequences or genetically altering cellular signaling pathways to increase production of the antigen. Also, cells can be transduced with a vector coding for the antigen or immunogenic fragment thereof.

By the term “systemic immune response” or grammatical equivalents herein is meant an immune response which is not localized, but affects the individual as a whole, thus allowing specific subsequent responses to the same stimulus.

As used herein, the term “proliferation-incompetent” or “inactivated” refers to cells that are unable to undergo multiple rounds of mitosis, but still retain the capability to express proteins such as cytokines or tumor antigens. This may be achieved through numerous methods known to those skilled in the art. Embodiments of the methods include, but are not limited to, treatments that inhibit at least about 95%, at least about 99% or substantially 100% of the cells from further proliferation. In one embodiment, the cells are irradiated at a dose of from about 50 to about 200 rads/min or from about 120 to about 140 rads/min prior to administration to the mammal. Typically, when using irradiation, the levels required are 2,500 rads, 5,000 rads, 10,000 rads, 15,000 rads or 20,000 rads. In several embodiments, the cells produce beta-filamin or immunogenic fragment thereof, two days after irradiation, at a rate that is at least about 10%, at least about 20%, at least about 50% or at least about 100% of the pre-irradiated level, when standardized for viable cell number. In one embodiment, cells are rendered proliferation incompetent by irradiation prior to administration to the subject.

By the term “individual”, “subject” or grammatical equivalents thereof is meant any one individual mammal.

By the term “reversal of an established tumor” or grammatical equivalents herein is meant the suppression, regression, or partial or complete disappearance of a pre-existing tumor. The definition is meant to include any diminution in the size, potency or growth rate of a pre-existing tumor.

The terms “treatment”, “therapeutic use”, or “medicinal use” as used herein, shall refer to any and all uses of the claimed compositions which remedy a disease state or symptom, or otherwise prevent, hinder, retard, or reverse the progression of disease or other undesirable symptoms in any way whatsoever.

The term “administered” refers to any method that introduces cells of a cancer immunotherapy described herein (e.g. genetically modified GM-CSF expressing cancer cells) to a mammal. This includes, but is not limited to, intradermal, parenteral, intramuscular, subcutaneous, intraperitoneal, intranasal, intravenous (including via an indwelling catheter), intratumoral, via an afferent lymph vessel, or by another route that is suitable in view of the patient's condition. The compositions provided herein may be administered to the subject at any site. For example, they can be delivered to a site that is “distal” to or “distant” from the primary tumor.

The term “increased immune response” as used herein means that a detectable increase of a specific immune activation is detectable (e.g. an increase in B-cell and/or T-cell response and/or NK cell response). An example of an increased immune response is an increase in the amount of an antibody that binds an antigen which is not detected or is detected a lower level prior to administration of a cytokine-expressing cellular immunotherapy provided herein. Another example, is an increased cellular immune response. A cellular immune response involves T cells, and can be observed in vitro (e.g. measured by a Chromium release assay) or in vivo. An increased immune response is typically accompanied by an increase of a specific population of immune cells.

By the term “retarding the growth of a tumor” is meant the slowing of the growth rate of a tumor, the inhibition of an increase in tumor size or tumor cell number, or the reduction in tumor cell number, tumor size, or numbers of tumors.

The term “inhibiting tumor growth” refers to any measurable decrease in tumor mass, tumor volume, amount of tumor cells or growth rate of the tumor. Measurable decreases in tumor mass can be detected by numerous methods known to those skilled in the art. These include direct measurement of accessible tumors, counting of tumor cells (e.g. present in blood), measurements of tumor antigens, Alphafetoprotein (AFP) and various visualization techniques (e.g. MRI, CAT-scan and X-rays). Decreases in the tumor growth rate typically correlates with longer survival time for a mammal with cancer.

By the term “therapeutically effective amount” or grammatical equivalents herein refers to an amount of an agent, e.g., a cytokine-expressing cellular immunotherapy provided herein, that is sufficient to modulate, either by stimulation or suppression, the immune response of an individual. This amount may be different for different individuals, different tumor types, and different preparations. The “therapeutically effective amount” is determined using procedures routinely employed by those of skill in the art such that an “improved therapeutic outcome” results.

As used herein, the terms “improved therapeutic outcome” and “enhanced therapeutic efficacy”, relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including an increase in life expectancy or an improvement in quality of life (as further described herein)

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). Nucleotides are indicated by their bases by the following standard abbreviations: adenine (A), cytosine (C), thymine (T), and guanine (G).

Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, New York. Generally, highly stringent hybridization and wash conditions are selected to be about 5° C. to 20° C. (preferably 5° C.) lower than the thermal melting point (Tₘ) for the specific sequence at a defined ionic strength and pH. Typically, under highly stringent conditions a probe will hybridize to its target subsequence, but to no other unrelated sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein or by visual inspection

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information, or by visual inspection (see generally, Ausubel et al., infra). For purposes of the compositions and methods provided herein, optimal alignment of sequences for comparison is most preferably conducted by the local homology algorithm of Smith & Waterman, Adv. Appl. Math. 2: 482 (1981).

As used herein, a “peptide” refers to an amino acid polymer containing between about 8 and about 12 amino acids linked together via peptide bonds. A peptide as used herein can comprise additional atoms beyond those of the 8 to twelve amino acids, so long as the peptide retains the ability to bind an MHC I receptor, e.g., an HLA-A2 receptor, and form a ternary complex with the T-cell receptor, the MHC I receptor, and the peptide.

Conservative substitution” refers to the substitution in a polypeptide of an amino acid with a functionally similar amino acid. The following six groups each contain amino acids that are conservative substitutions for one another:
Alanine (A), Serine (S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W).

The term “about,” as used herein, unless otherwise indicated, refers to a value that is no more than 10% above or below the value being modified by the term. For example, the term “about 5 μg/kg” means a range of from 4.5 μg/kg to 5.5 μg/kg. As another example, “about 1 hour” means a range of from 48 minutes to 72 minutes. Where the term “about” modifies a value that must be an integer, and 10% above or below the value is not also an integer, the modified value should be rounded to the nearest whole number. For example, “about 12 amino acids” means a range of 11 to 13 amino acids.

The term “physiological conditions,” as used herein, refers to the salt concentrations normally observed in human serum. One skilled in the art will recognize that physiological conditions need not mirror the exact proportions of all ions found in human serum, rather, considerable adjustment can be made in the exact concentration of sodium, potassium, calcium, chloride, and other ions, while the overall ionic strength of the solution remains constant.

### 4.2 ANTIGENS ASSOCIATED WITH THERAPY WITH PROLIFERATION INCOMPETENT TUMOR CELLS THAT EXPRESS GM-CSF

In certain aspects as described below, provided herein are methods that comprise assessing immune responses against antigens associated with a likelihood of responsiveness to treatment with proliferation-incompetent tumor cells that express cytokines, e.g., GM-CSF. In some embodiments, the therapies are predicted to result in an improved therapeutic outcome for the subject, for example, a decrease in cancer-associated pain or improvement in the condition of the patient according to any clinically acceptable criteria, including but not limited to a decrease in metastases, an increase in life expectancy or an improvement in quality of life. The antigens may be expressed endogenously by cells native to the subject or may be exogenously provided to the subject by, e.g., the administered engineered tumor cells. The discussion below briefly describes examples of such antigens.

Coatomer binding complex (beta′-coat protein; coatomer subunit beta′; coatomer binding complex, beta prime subunit; coatomer protein complex, subunit beta; coatomer protein complex, subunit beta 2 (beta prime)) is one of the subunits of an oligomeric complex putatively involved in regulating membrane trafficking in the exocytic pathway. Coatomer binding complex (aliases include COPB2, Beta′-COP, beta′-COP, and p102) encodes a 102487 Da protein comprised of 906 amino acids (REFSEQ NP_{—}004757.1, SEQ ID NO: 1) that is encoded on chromosome 3 (Ensembl cytogenetic band: 3q23). A representative nucleotide sequence is NM_{—}004766.1 (SEQ ID NO: 2). COPB2 is part of a cytosolic protein complex constitutes the coat of nonclathrin-coated vesicles and is essential for Golgi budding and vesicular trafficking (Stenbeck et al., EMBO J. 12, 2841-2845 (1993); Harrison-Lavoie et al., EMBO J. 12, 2847-2853 (1993). Meta-analysis of DNA microarray data revealed that COPB2 is commonly activated in human cancer relative to respective normal tissue types (Rhodes and Chinnaiyan, Nat. Genet. 37, S31-37 (2005).

Glutamyl-prolyl tRNA synthetase (bifunctional aminoacyl-tRNA synthetase; multifunctional aminoacyl-tRNA synthetase; glutamate tRNA ligase; glutaminyl-tRNA synthetase; glutamyl-prolyl tRNA synthetase; proliferation-inducing protein; proline-tRNA ligase; prolyl-tRNA synthetase) is a component of the multisynthetase complex which is comprised of a bifunctional glutamyl-prolyl-tRNA synthetase, the monospecific isoleucyl, leucyl, glutaminyl, methionyl, lysyl, arginyl, and aspartyl-tRNA synthetases as well as three auxiliary proteins, p18, p48 and p43. Aliases for glutamyl-prolyl tRNA synthetase include EPRS, DKFZp313B047, EARS, GLNS, PARS, PIG32, QARS, and QPRS. EPRS encodes a 163026 Da protein comprised of 1440 amino acids (REFSEQ NP_{—}004437.2, SEQ ID NO: 3) that is encoded on chromosome 1 (Ensembl cytogenetic band: 1q41). A representative nucleotide sequence is NM_{—}004446.2 (SEQ ID NO: 4). Aminoacyl-tRNA synthetases are a class of enzymes that charge tRNAs with their cognate amino acids (Hirano, Arterioscler. Thromb. Vasc. Biol. 27, 27-36 (2007). The protein encoded by this gene is a multifunctional aminoacyl-tRNA synthetase that catalyzes the aminoacylation of glutamic acid and proline tRNA species (Fett and Knippers, J. Biol. Chem. 266, 1448-1455 (1991); Cerini et al., EMBO J. 10, 4267-4277 (1991)). Alternative splicing has been observed for this gene, but the full-length nature and biological validity of the variant have not been determined. Serological responses to EPRS have previously been observed in colon cancer patients (Line et al., Cancer Immunol. Immunother. 51, 574-582 (2002).

DEAD (Asp-Glu-Ala-Asp) box polypeptide 41 (DEAD box protein 41; DEAD box protein abstrakt homolog; DEAD-box protein abstract; probable ATP-dependent RNA helicase DDX41; putative RNA helicase) is a member of a diverse family of nuclear proteins involved in ATP-dependent RNA unwinding, needed in a variety of cellular processes including splicing, ribosome biogenesis and RNA degradation. Aliases for this gene include DDX41, 2900024F02Rik; ABS; EC 3.6.1.-; and MGC8828. The DDX41 gene encodes for a 622 amino acid protein of 69838 Da (REFSEQ NP_{—}057306.2, SEQ ID NO: 5). The gene is located on chromosome 5 (Ensembl cytogenetic band: 5q35.3), and a representative nucleotide sequence is NM_{—}016222.2 (SEQ ID NO: 6). DEAD box proteins, characterized by the conserved motif Asp-Glu-Ala-Asp (DEAD), are putative RNA helicases. They are implicated in a number of cellular processes involving alteration of RNA secondary structure, such as translation initiation, nuclear and mitochondrial splicing, and ribosome and spliceosome assembly. Based on their distribution patterns, some members of the DEAD box protein family are believed to be involved in embryogenesis, spermatogenesis, and cellular growth and division. This gene encodes a member of this family. The function of this member has not been determined. Based on studies in Drosophila, the abstract gene is widely required during post-transcriptional gene expression. See Irion and Leptin, Curr. Biol. 9, 1373-1381 (1999); Abdelhaleem, Clin. Biochem. 38, 499-503 (2005); Abdul-Ghani et al., J. Cell Physiol. 204, 210-218 (2005).

Interleukin-1 receptor associated kinase 4 (IRAK-4 mutated form; NY-REN-64 antigen 3) is required for the efficient recruitment of IRAK1 to the IL-1 receptor complex following IL-1 engagement, triggering intracellular signaling cascades leading to transcriptional up-regulation and mRNA stabilization. Common aliases for interleukin-1 receptor associated, kinase 4 include IRAK4, EC 2.7.11.1, IPD1, LOC51135, NY-REN-64, and REN64. The IRAK4 gene encodes for a 460 amino acid protein of 51530 Da (REFSEQ NP_{—}057207.1, SEQ ID NO: 7). The gene is located on chromosome 12 (Ensembl cytogenetic band: 12q12), and a representative nucleotide sequence is NM_{—}016123.1 (SEQ ID NO: 8). The interleukin-1 receptor associated kinases (e.g., IRAK4) are important mediators in the signal transduction of Toll-like receptor (TLR) and IL1R family members. Thus IRAK-4 plays a critical role in IL-1 receptor (IL-1R)/TLR7-mediated induction of inflammatory responses. See Burns et al., J. Exp. Med. 20, 197, 263-268 (2003); Suzuki et al., Science 311, 1927-1932 (2006); Koziczak-Holbro et al., J. Biol. Chem. 282, 13552-13560 (2007); Li et al., Proc. Natl. Acad. Sci. U.S.A. 99, 5567-5572 (2002).

Cytosolic malate dehydrogenase (malate dehydrogenase, cytoplasmic; malate dehydrogenase 1, NAD (soluble); soluble malate dehydrogenase) is important in transporting NADH equivalents across the mitochondrial membrane, controlling tricarboxylic acid (TCA) cycle pool size and providing contractile function (Lo et al., J. Cell Biochem. 94, 763-773 (2005). The synonyms for cytosolic malate dehydrogenase include MDH1, MDHA, MOR2, MDH-s, MGC:1375, and EC 1.1.1.37. MDH1 encodes a 36426 Da cytoplasmic protein comprised of 334 amino acids (REFSEQ NP_{—}005908.1, SEQ ID NO: 9). The genomic location of MDH1 is chromosome 2 (Ensembl cytogenetic band: 2p15), and a representative nucleotide sequence is NM_{—}005917.2 (SEQ ID NO: 10). Malate dehydrogenase catalyzes the reversible oxidation of malate to oxaloacetate, utilizing the NAD/NADH cofactor system in the citric acid cycle. The protein encoded by this gene is localized to the cytoplasm and may play pivotal roles in the malate-aspartate shuttle that operates in the metabolic coordination between cytosol and mitochondria (Friedrich et al., Biochem. Genet. 25, 657-669 (1987); Friedrich et al., Ann Hum Genet. 52, 25-37 (1988)). MDH1 was found to be overexpressed in null cell adenomas compared to normal pituitary by expressed sequence tag sequencing and cDNA microarray analysis (Hu et al., Pituitary 10, 47-52 (2007).

The H1 histone family, member 2 (histone H1.2; histone H1d; histone 1, H1c; histone cluster 1, H1c) is also referred to by the aliases HIST1H1C, H1.2, H1F2, and MGC3992. Histones are necessary for the condensation of nucleosome chains into higher order structures. HIST1H1C encodes a nuclear protein 21365 Da in size encoded by 213 amino acids (REFSEQ NP_{—}005310.1, SEQ ID NO: 11). The genomic location of HIST1H1C is chromosome 6 (Ensembl cytogenetic band: 6p22.2), and a representative nucleotide sequence is NM_{—}005319.3 (SEQ ID NO: 12). Histones are basic nuclear proteins responsible for nucleosome structure of the chromosomal fiber in eukaryotes. Two molecules of each of the four core histones (H2A, H2B, H3, and H4) form an octamer, around which approximately 146 by of DNA is wrapped in repeating units, called nucleosomes. The linker histone, H1, interacts with linker DNA between nucleosomes and functions in the compaction of chromatin into higher order structures. This gene is intronless and encodes a member of the histone H1 family. Transcripts from this gene lack polyA tails but instead contain a palindromic termination element. This gene is found in the large histone gene cluster on chromosome 6. See Ohe et al., J. Biochem. 106, 844-857 (1989); Eick et al., Eur. J. Cell Biol. 49, 110-115 (1989).

Zuotin related factor 1 (DnaJ homolog subfamily C member 2; m-phase phosphoprotein 11) is also known by the synonyms ZRF1, zuotin, ZUO1, MPP11, MPHOSPH11, and DNAJC2. ZRF1 is comprised of 621 amino acids, and encodes for a 71897 Da protein (REFSEQ NP_{—}055192.1, SEQ ID NO: 13). A representative nucleotide sequence is NM_{—}014377.1 (SEQ ID NO: 14), located on chromosome 7 (Ensembl cytogenetic band: 7q22.1). ZRF1 is a member of the M-phase phosphoprotein (MPP) family, and encodes a phosphoprotein with a J domain and a Myb DNA-binding domain which localizes to both the nucleus and the cytosol. The protein is capable of forming a heterodimeric complex that associates with ribosomes, acting as a molecular chaperone for nascent polypeptide chains as they exit the ribosome. This protein was identified as a leukemia-associated antigen and expression of the gene is upregulated in leukemic blasts. Also, chromosomal aberrations involving this gene are associated with primary head and neck squamous cell tumors. This gene has a pseudogene on chromosome 6. Alternatively spliced variants which encode different protein isoforms have been described; however, not all variants have been fully characterized. See Otto et al., Proc. Natl. Acad. Sci. U.S.A. 102, 10064-10069 (2005); Greiner et al., Int. J. Cancer 108, 704-711 (2004); Greiner et al., Int. J. Cancer 106, 224-231 (2003); Resto et al., Cancer Res. 60, 5529-5535 (2000); Matsumoto-Taniura et al., Mol. Biol. Cell 7, 1455-1469 (1996).

DNA topoisomerase 2-beta (DNA topoisomerase II beta; DNA topoisomerase II, 180 kD; DNA topoisomerase II, beta isozyme; U937 associated antigen; antigen MLAA-44) is a ubiquitous ATPase component of the topoisomerase II involved in the breakage and rejoining of double strand of DNA. Aliases for this gene include TOP2B, TOPIIB, top2beta, and EC 5.99.1.3. TOP2B is comprised of 1626 amino acids, and encodes for a 183267 Da protein (REFSEQ NP_{—}001059.2, SEQ ID NO: 15). A representative nucleotide sequence is NM_{—}001068.2 (SEQ ID NO: 16), located on chromosome 3 (Ensembl cytogenetic band: 3p24.2). TOP2B encodes a DNA topoisomerase, an enzyme that controls and alters the topologic states of DNA during transcription. This nuclear enzyme is involved in processes such as chromosome condensation, chromatid separation, and the relief of torsional stress that occurs during DNA transcription and replication. It catalyzes the transient breaking and rejoining of two strands of duplex DNA which allows the strands to pass through one another, thus altering the topology of DNA. Two forms of this enzyme exist as likely products of a gene duplication event. The gene encoding this form, beta, is localized to chromosome 3 and the alpha form is localized to chromosome 17. The gene encoding this enzyme functions as the target for several anticancer agents and a variety of mutations in this gene have been associated with the development of drug resistance. Reduced activity of this enzyme may also play a role in ataxia-telangiectasia. Alternative splicing of this gene results in two transcript variants; however, the second variant has not yet been fully described. See Mimeault et al., Int. J. Cancer. 120, 160-9 (2007); Chikamori et al., Leukemia 20, 1809-1818 (2006); Austin et al., Biochim. Biophys. Acta 1172, 283-291 (1993); Jenkins et al., Nucleic Acids Res. 20, 5587-5592 (1992); Tan et al., Cancer Res. 52, 231-234 (1992); Austin and Fisher, FEBS Lett. 266, 115-117 (1990); Chung et al., Proc Natl. Acad. Sci. U.S.A. 86, 9431-9435 (1989).

A kinase (PRKA) anchor protein (A-kinase anchor protein 350 kDa; A-kinase anchor protein 450 kDa; centrosome- and golgi-localized PKN-associated protein; protein hyperion; protein yotiao; AKAP9-BRAF fusion protein; AKAP120-like protein) has a number of aliases, including AKAP9, AKAP350, AKAP450, CG-NAP, HYPERION, KIA0803, and yotiao. AKAP9 may be required to maintain the integrity of the Golgi apparatus, and one of the isoforms of may play a role in the organization of postsynaptic specializations. There are at least 6 isoforms of AKAP9 produced by alternative splicing, including REFSEQ NP_{—}005742.4, NP_{—}671695.1, NP_{—}671700.1, NP_{—}671714.1 (SEQ ID NOS: 17-20). The consensus sequence of AKAP9 is comprised of 3911 amino acids, and encodes for a 453667 Da protein. Representative nucleotide sequences for some of the isoforms are NM_{—}005751.3, NM_{—}147166.1, NM_{—}147171.1, NM_{—}147185.1 (SEQ ID NOS: 21-24), located on chromosome 7 (Ensembl cytogenetic band: 7q21.2). The A-kinase anchor proteins (AKAPs) are a group of structurally diverse proteins which have the common function of binding to the regulatory subunit of protein kinase A (PKA) and confining the holoenzyme to discrete locations within the cell. Alternate splicing of this gene results in many isoforms that localize to the centrosome and Golgi apparatus. These isoforms interact with numerous signaling proteins from multiple signal transduction pathways, including type II protein kinase A, serine/threonine kinase protein kinase N, protein phosphatase 1, protein phosphatase 2a, protein kinase C-epsilon and phosphodiesterase 4D3. Oncogenic fusions between AKAP9 and BRAF have also been observed in thyroid papillary carcinomas. See Westphal et al., Science 285, 93-96 (1999); Takahashi et al., J. Biol. Chem. 274, 17267-17274 (1999); Witczak et al., EMBO J. 18, 1858-1868 (1999); Schmidt et al., J. Biol. Chem. 274, 3055-3066 (1999); Lin et al., J. Neurosci. 18, 2017-2027 (1998); E1 Din El Homasany et al., J. Immunol. 175, 7811-7818 (2005); Clampi et al., J. Clin. Invest. 115, 20-23 (2005).

Transmembrane protein 33 is also know by the aliases TMEM33 and DB83, and was identified by tandem mass spectrometry of melanosome proteomes at various developmental stages (Chi et al., J Proteome Res. 5, 3135-3144 (2006); Ewing et al., Mol Syst Biol. 3, 89 (2007)). The function of TMEM33 is not known. The TMEM33 gene encodes for a 247 amino acid protein of 27978 Da (REFSEQ NP_{—}060596.1, SEQ ID NO: 25). The gene is located on chromosome 4 (Ensembl cytogenetic band: 4p13), and a representative nucleotide sequence is NM_{—}018126.1 (SEQ ID NO: 26).

SET domain containing 1B (SET domain-containing protein 1B -Fragment SETD1B) is also known by the synonyms SETD1B, F1120803, and KIAA1076. SETD1B was derived by automated computational analysis using the GNOMON gene prediction method with supporting evidence based on sequence similarity to 4 mRNAs, 94 ESTs, and 6 proteins (Kikuno et al., DNA Res. 6, 197-205 (1999)). The gene product of SETD1B is similar to SET domain containing 1A and has no known function. The SETD1B gene encodes for a 2037 amino acid protein of 221106 Da (REFSEQ XP_{—}037523.11, SEQ ID NO: 27). The gene is located on chromosome 12 (Ensembl cytogenetic band: 12q24.31), and a representative nucleotide sequence is XM_{—}037523 (SEQ ID NO: 28).

The first third of B double prime 1, subunit of RNA polymerase III transcription initiation factor IIIB (BDP1; mRNAB double prime 1, subunit of RNA polymerase III transcription initiation factor IIIB; TFC5; TFNR; TAF3B1; KIAA1241; KIAA1689; TFIIIB; TFIIIB90; HSA238520; TFIIIB150; DKFZp686K0831; DKFZp686C01233; transcription factor-like nuclear regulator; TATA box binding protein (TBP)-associated factor; RNA polymerase III, GTF3B subunit 1; transcription factor IIIB 150; RNA polymerase III transcription initiation factor B″) encodes a subunit of the RNA polymerase III (Pol III) transcription factor complex. The BDP1 gene encodes for a 2624 amino acid protein of 293755 Da (REFSEQ NP_{—}060899.2, SEQ ID NO: 29). The gene is located on chromosome 5 (Ensembl cytogenetic band: 5q13.2), and a representative nucleotide sequence is NM_{—}018429.2 (SEQ ID NO: 30). The product of this gene is a subunit of the TFIIIB transcription initiation complex, which recruits RNA polymerase III to target promoters in order to initiate transcription. The encoded protein localizes to concentrated aggregates in the nucleus, and is required for transcription from all three types of polymerase III promoters. It is phosphorylated by casein kinase II during mitosis, resulting in its release from chromatin and suppression of polymerase III transcription. See Johnson et al., Mol. Cell. 26, 367-379 (2007); Schoenen et al., Biol. Chem. 387, 277-284 (2006); Hu et al., Mol. Cell. 16, 81-92 (2004); Weser et al., J. Biol. Chem. 279, 27022-27029 (2004); Fairley et al., EMBO J. 22, 5841-5850 (2003); Hu et al., Mol. Cell. 12, 699-709 (2003); Kelter et al., Genomics 70, 315-326 (2000); Schramm et al., Genes Dev. 14, 2650-2663 (2000); Chu et al., J. Biol. Chem. 272, 14755-14761 (1997); Wang et al., Genes Dev. 11, 1315-1326 (1997).

Centrosomal protein 290 kDa (CTCL tumor antigen se2-2; prostate cancer antigen T21; nephrocystin 6; monoclonal antibody 3H11 antigen; nephrocytsin-6; Joubert syndrome 5; nephrocystin-6) is required for the correct localization of ciliary and phototransduction proteins in retinal photoreceptor cells, and may play a role in ciliary transport processes. Synonyms for centrosomal protein 290 kDa include MKS4, rd16, JBTS5, JBTS6, LCA10, NPHP6, SLSN6, 3H11Ag, FLJ13615, FLJ21979, and KIAA0373. CEP290 encodes a 290386 Da protein comprised of 2479 amino acids (REFSEQ NP_{—}079390.3, SEQ ID NO: 31) that is encoded on chromosome 12 (Ensembl cytogenetic band: 12q21.32). A representative nucleotide sequence is NM_{—}025114.3 (SEQ ID NO: 32). CEP290 encodes a protein with 13 putative coiled-coil domains, a region with homology to SMC chromosome segregation ATPases, six KID motifs, three tropomyosin homology domains, and an ATP/GTP binding site motif A. The protein is localized to the centrosome and cilia and has sites for N-glycosylation, tyrosine sulfation, phosphorylation, N-myristoylation, and amidation. Mutations in this gene have been associated with Joubert syndrome and nephronophthisis and the presence of antibodies against this protein is associated with several forms of cancer. See Perrault et al., Hum. Mutat. 28, 416 (2007); Olsen et al., Cell 127, 635-648 (2006); den Hollander et al., Am. J. Hum. Genet. 79, 556-561 (2006); Sayer et al., Nat. Genet. 38, 674-681 (2006); Valente et al., Nat. Genet. 38, 623-625 (2006); Guo et al., Biochem. Biophys. Res. Commun. 324, 922-930 (2004); Andersen et al., Nature 426, 570-574 (2003); Shin et al., J. Biol. Chem. 278, 7607-7616 (2003); Eichmuller et al., Proc. Natl. Acad. Sci. U.S.A. 98, 629-634 (2001); Chen et al., Biochem. Biophys. Res. Commun. 280, 99-103 (2001).

AHNAK nucleoprotein 2 (AHNAK nucleoprotein (desmoyokin); neuroblast differentiation-associated protein AHNAK) is also known by the aliases AHNAK, AHNAKRS, and desmoyokin. AHNAK is a ubiquitously expressed giant phosphoprotein that was initially identified as a gene product subject to transcriptional repression in neuroblastoma. The AHNAK gene is located on chromosome 11 (Ensembl cytogenetic band: 11q12.3), and encodes for at least two transcripts whose representative nucleotide sequences are NM_{—}001620.1 (SEQ ID NO: 33) and NM_{—}024060.2 (SEQ ID NO: 34). The larger of these transcripts encodes for a 5890 amino acid protein of 628973 Da (REFSEQ NP_{—}001611, SEQ ID NO: 35). AHNAK encodes an unusually large protein that is expressed by means of a 17.5-kilobase mRNA in diverse cellular lineages, but is typically repressed in cell lines derived from human neuroblastomas and in several other types of tumors. AHNAK is implicated in calcium flux regulation and has emerged as an important signaling molecule in a wide range of physiological activities. AHNAK is critical for cardiac Ca(V)1.2 calcium channel function and its beta-adrenergic regulation. AHNAK was also identified as a potential diagnostic marker for ovarian cancer. See De Seranno et al., J. Biol. Chem. 281, 35030-35038 (2006); Haase et al., FASEB J. 19, 1969-1977 (2005); Lee et al., J. Biol. Chem. 279, 26645-26653 (2004); Sussman et al., J. Cell. Biol. 154, 1019-1030 (2001); Gentil et al., J. Biol. Chem. 276, 23253-23261 (2001); Kudoh et al., Cytogenet. Cell Genet. 70, 218-220 (1995); Shtivelman et al., Proc. Natl. Acad. Sci. U.S.A. 89, 5472-5476 (1992); Chatterjee et al., Cancer Res. 66, 1181-1190 (2006).

PDGFA associated protein 1 (PDGF associated protein) is also known by the aliases PDAP1, PAP, PAP1, and HASPP28. The PDAP1 gene encodes for a 181 amino acid protein of 20630 Da (REFSEQ NP_{—}055706.1, SEQ ID NO: 36). The gene is located on chromosome 7 (Ensembl cytogenetic band: 7q22.1), and a representative nucleotide sequence is NM_{—}014891.5 (SEQ ID NO: 37). PDAP1 is a novel mitogen-associated protein that was isolated from a rat neural retina cell line. The protein co-purified with platelet-derived growth factor (PDGF)-A. PDAP1 binds to PDGF with low affinity and enhances the mitogenic effect of PDGF-A, but lowers the mitogenic activity of PDGF-B. PDAP1 is expressed in the brain of newborn rats and is found in several other tissues. See LaRochelle et al., Nat. Cell Biol. 3, 517-521 (2001); Fischer et al., J. Neurochem. 66, 2213-2216 (1996).

Leucine zipper and CTNNBIP1 domain-containing protein (LZIC; leucine zipper and ICAT homologous domain-containing protein; MGC15436) encodes for a 191 amino acid protein of 21495 Da (REFSEQ NP_{—}115744.2, SEQ ID NO: 38). The gene is located on chromosome 1 (Ensembl cytogenetic band: 1p36.22), and a representative nucleotide sequence is NM_{—}032368.3 (SEQ ID NO: 39). The major 5.2-kb LZIC mRNA and minor 2.1-, 1.6-, and 1.0-kb LZIC mRNAs are expressed almost ubiquitously in normal human tissues. LZIC is also expressed in numerous cancer cell lines, and is significantly up-regulated in the gastric cancer cell line MKN74 and 5 cases of primary gastric cancer. As LZIC contains ICAT homologous domain, LZIC might inhibit the interaction between beta-catenin and TCF transcription factors, and up-regulation of LZIC in gastric cancer might be due to a negative feed-back mechanism to inhibit the WNT-beta-catenin-TCF signaling pathway. See Katoh et al., Int. J. Mol. Med. 8, 611-615 (2001).

Zinc finger protein 397 (zinc finger and SCAN domain-containing protein 15; zinc finger protein 47) is also known by the synonyms ZNF397, 2810411K16Rik, ZNF47, and ZSCAN15, and MGC13250. ZNF397 encodes a 61139 Da protein comprised of 534 amino acids (REFSEQ NP_{—}115723.1, SEQ ID NO: 40) that is encoded on chromosome 18 (Ensembl cytogenetic band: 18q12.2). A representative nucleotide sequence is NM_{—}032347.1 (SEQ ID NO: 41). Four isoforms of ZNF397 transcript, 1.7, 2.5, 7.0 and 9.0-kb, are expressed in a variety of tissues, with varying levels. The SCAN-(C(2)H(2))(X) genes encode two distinct proteins due to a unique alternative splicing mechanism. ZNF397-fu (full zinc fingers) consists of a SCAN domain in the N-terminal region and many consecutive C(2)H(2) zinc finger repeats in the C-terminal region. ZNF397-nf (no zinc fingers) encodes 198 amino acids containing the SCAN domain only. ZNF397-fu or ZNF397-nf can homo-associate, and ZNF397-fu hetero-associates with ZNF397-nf. ZNF397-nf polypeptides are expressed diffusely in the cells, while ZNF397-fu polypeptides target specifically to the nuclei. ZNF397-nf can repress reporter gene transcription, with ZNF397-nf having the strongest repression activity. Deletion analysis revealed that ZNF397-fu is a transcriptional activator without its nine zinc finger repeats. See Wu et al., Gene 310, 193-201 (2003); Lichter et al., Genomics 13, 999-1007 (1992).

Structural maintenance of chromosomes 1A (structural maintenance of chromosomes 1, yeast-like 1; segregation of mitotic chromosomes 1; SMC1 alpha protein) is also known by the synonyms SMC1A, SMC1, SMCB, CDLS2, SB1.8, SMC1L1, DXS423E, KIAA0178, MGC138332, SMC1alpha, and DKFZp686L19178. Proper cohesion of sister chromatids is a prerequisite for the correct segregation of chromosomes during cell division. The cohesin multiprotein complex is required for sister chromatid cohesion. This complex is composed partly of two structural maintenance of chromosomes (SMC) proteins, SMC3 and either SMC1L2 or the protein encoded by this gene. Most of the cohesin complexes dissociate from the chromosomes before mitosis, although those complexes at the kinetochore remain. Therefore, the encoded protein is thought to be an important part of functional kinetochores. In addition, this protein interacts with BRCA1 and is phosphorylated by ATM, indicating a potential role for this protein in DNA repair. This gene, which belongs to the SMC gene family, is located in an area of the X-chromosome that escapes X inactivation. SMC1A is comprised of 1233 amino acids, and encodes for a 143233 Da protein (REFSEQ NP_{—}006297.2, SEQ ID NO: 42). A representative nucleotide sequence is NM_{—}006306.2 (SEQ ID NO: 43), located on chromosome X (Ensembl cytogenetic band: Xp11.22). Defects in SMC are the cause of X-linked Cornelia de Lange syndrome [MIM:300590]. Cornelia de Lange syndrome is a clinically heterogeneous developmental disorder associated by malformations affecting multiple systems. Cornelia de Lange is characterized by facial dysmorphisms, upper limb abnormalities, growth delay and cognitive retardation. Mutations in the NIPBL gene, a component of the cohesin complex, account for approximately half of the affected individuals. Mutations in the SMC1A, which encodes a different subunit of the cohesin complex, are responsible for an X-linked form of the disorder. See Deardorff et al., Am. J. Hum. Genet. 80, 485-494 (2007); Schoumans et al., Eur. J. Hum. Genet. 15, 143-149 (2007); Inoue et al., Biochem. J. 398, 125-133 (2006); Musio et al., Nat. Genet. 38, 528-530 (2006); Ryu et al., Biochem. Biophys. Res. Commun. 341, 770-775 (2006); Yazdi et al., Genes Dev. 16, 571-582 (2002); Schmiesing et al., Proc. Natl. Acad. Sci. U.S.A. 95 (22), 12906-12911 (1998).

The MYO18A gene has previously been described as molecule associated with JAK3 N-terminus, myosin containing a PDZ domain, SP-A receptor subunit SP-R210 alphaS, myosin 18A, myosin XVIIIA, myosin containing PDZ domain. Aliases for MYO18A include DKFZp686L0243, MAJN, MYSPDZ, MysPDZ, SPR210, myosin XVIIIA, KIAA0216, and TIAF1. There are two isoforms of MYO18A, one that is localized to the endoplasmic reticulum-golgi intermediate compartment (by similarity), and a second isoform that is localized to the cytoplasm. Isoform 1 colocalizes with actin, whereas isoform 2 lacks the PDZ domain and is diffusely localized in the cytoplasm. The amino acid sequences for these isoforms are REFSEQ NP_{—}510880.2 (SEQ ID NO: 44) and REFSEQ NP_{—}976063.1 (SEQ ID NO: 45). MYO18A is encoded on chromosome 17 (Ensembl cytogenetic band: 17q11.2), and representative nucleotide sequences are NM_{—}078471.3 (SEQ ID NO: 46) and NM_{—}203318.1 (SEQ ID NO: 47). MYO18A is an unconventional myosin belonging to the class XVIII myosin containing a KE (lysine and glutamine)-rich domain and a PDZ domain, which codistributes with actin fibers partially without any canonical actin binding sequence in its myosin head domain. Thus this gene appears to be involved in the maintenance of the stromal cell architectures required for cell to cell contact. See Mori et al., J. Biochem. 133, 405-413 (2003); Ji et al., Biochem. Biophys. Res. Commun. 270, 267-271 (2000); Chang et al., Biochem. Biophys. Res. Commun. 253, 743-749 (1998); Chroneos et al., J. Biol. Chem. 271, 16375-16383 (1996); Yang et al., J. Biol. Chem. 280, 34447-34457 (2005); Kim et al., J. Proteome Res. 4, 1339-1346 (2005); Isogawa et al., Biochemistry 44, 6190-6196 (2005); Homma et al., J. Mol. Biol. 343 (5), 1207-1220 (2004).

The PALLD gene has been described as PALLD protein-fragment 4; pancreatic cancer, susceptibility to; palladin, cytoskeletal associated protein; and sarcoma antigen NY-SAR-77 and has numerous synonyms, including CGI-151, FLJ22190, FLJ38193, FLJ39139, KIAA0992, PNCA1, SIH002, and palladin. PALLD is comprised of 510 amino acids, and encodes for a 57061 Da protein (REFSEQ NP_{—}057165.3, SEQ ID NO: 48). A representative nucleotide sequence is NM_{—}016081.3 (SEQ ID NO: 49), located on chromosome 4 (Ensembl cytogenetic band: 4q32.3). PALLD is a major component of stress fiber dense bodies, cardiomyocyte Z-discs, and neuronal synapses. It functions as a structural molecule, cytoskeletal regulator, and docking site to other proteins. Both antisense and transient overexpression experiments have shown that PALLD plays an important role in the regulation of actin cytoskeleton. The function of PALLD is context dependent and plays a critical role in cytoskeletal remodeling, for example responding to signals induced by vascular injury as well as signals that induce smooth muscle cell hypertrophy, such as angiotension II. PALLD RNA was overexpressed in the tissues from precancerous dysplasia and pancreatic adenocarcinoma in both the familial and the sporadic disease. See Jin et al., Circ. Res. 100, 817-825 (2007); Pogue-Geile et al., PLoS Med. 3, E516 (2006); Boukhelifa et al., FEBS J. 273, 26-33 (2006); Ronty et al., Exp. Cell Res. 310, 88-98 (2005); Eberle et al., Am. J. Hum. Genet. 70, 1044-1048 (2002); Mykkanen et al., Mol. Biol. Cell 12, 3060-3073 (2001); Bang et al., J. Cell Biol. 153, 413-427 (2001); Parast and Otey, J. Cell Biol. 150, 643-656 (2000).

Spindle assembly abnormal protein is also known by the aliases SASS6, DKFZp761A078, FLJ22097, HsSAS-6, MGC119440, and SAS6. SASS6 is a coiled-coil protein that is recruited to centrioles at the onset of the centrosome duplication cycle, and is required for daughter centriole formation. SASS6 is comprised of 657 amino acids, and encodes for a 74397 Da protein (REFSEQ NP_{—}919268.1, SEQ ID NO: 50). A representative nucleotide sequence is NM_{—}194292.1 (SEQ ID NO: 51), located on chromosome 1 (Ensembl cytogenetic band: 1p21.2). See Leidel et al., Nat. Cell Biol. 7, 115-125 (2005); Dammermann et al., Dev. Cell 7, 815-829; Andersen et al., Nature 426, 570-574 (2003).

Phosphoserine aminotransferase 1 is also known by the synonyms PSAT1, EC 2.6.1.52, MGC1460, PSA, and PSAT. PSAT1 is the second step-catalyzing enzyme in the serine biosynthetic pathway in mammals and catalyzes the formation of phosphoserine from phosphohydroxypyruvate. The consensus PSAT1 sequence is comprised of 370 amino acids, and encodes for a 40423 Da protein. Two different isoforms of PSAT1 have been identified, alpha and beta, which differ in that PSAT alpha lacks an internal exon, but maintains the same reading frame. Reference amino acid sequences for these proteins are REFSEQ NP_{—}478059.1 (SEQ ID NO: 52) and REFSEQ NP_{—}066977.1 (SEQ ID NO: 53). Representative nucleotide sequences are NM_{—}058179.2 (SEQ ID NO: 54) and NM_{—}021154.3 (SEQ ID NO: 55), located on chromosome 9 (Ensembl cytogenetic band: 9q21.31). See Baek et al., Biochem. J. 373, 191-200 (2003); Basurko et al., IUBMB Life 48, 525-529 (1999); Misrahi et al., Biochemistry 26, 3975-3982 (1987).

Valosin-containing protein (VCP) is a member of a family that includes putative ATP-binding proteins involved in vesicle transport and fusion, 26S proteasome function, and assembly of peroxisomes. VCP, as a structural protein, is associated with clathrin, and heat-shock protein Hsc70, to form a complex. VCP has been implicated in a number of cellular events that are regulated during mitosis, including homotypic membrane fusion, spindle pole body function, and ubiquitin-dependent protein degradation.valosin-containing protein. VCP has also been described as yeast Cdc48p homolog and transitional endoplasmic reticulum ATPase, as well as the aliases IBMPFD, MGC1311997, MGC148092, MGC8560, TERA, and p97. VCP is comprised of 806 amino acids, and encodes for a 89322 Da protein (REFSEQ NP_{—}009057.1, SEQ ID NO: 56). A representative nucleotide sequence is NM_{—}007126.2 (SEQ ID NO: 57), located on chromosome 9 (Ensembl cytogenetic band: 9p13.3). VCP is necessary for the fragmentation of Golgi stacks during mitosis and for their reassembly after mitosis. VCP is also involved in the formation of the transitional endoplasmic reticulum (tER). The transfer of membranes from the endoplasmic reticulum to the Golgi apparatus occurs via 50-70 nm transition vesicles which derive from part-rough, part-smooth transitional elements of the endoplasmic reticulum (tER). Vesicle budding from the tER is an ATP-dependent process. The ternary complex containing UFD1L, VCP and NPLOC4 binds ubiquitinated proteins and is necessary for the export of misfolded proteins from the ER to the cytoplasm, where they are degraded by the proteasome. The NPLOC4-UFD 1 L-VCP complex appears to regulate spindle disassembly at the end of mitosis and is necessary for the formation of a closed nuclear envelope. See Zhang et al., Biochem. Biophys. Res. Commun. 356, 536-541 (2007); Rothballer et al., FEBS Lett. 581, 1197-1201 (2007); Qiu et al., Am. J. Pathol. 170, 152-159 (2007); Wojcik et al., Mol. Biol. Cell 17, 4606-4618 (2006); Mimnaugh et al., Mol. Cancer. Res. 4, 667-681 (2006); Zhang et al., J. Biol. Chem. 274, 17806-17812 (1999); Hoyle et al., Mamm. Genome 8, 778-780 (1997); Druck et al., Genomics 30, 94-97 (1995); Pleasure et al., Nature 365 (6445), 459-462 (1993).

Aliases for bromodomain adjacent to zinc finger domain 2B include BAZ2B, WALp4, FLJ45644, DKFZP434H071, DKFZp76210516, KIAA1476, and FLJ45644. BAZ2B encodes a 1972 amino acid protein of 220710 Da in size (REFSEQ NP_{—}038478.2, SEQ ID NO: 58). The genomic location of BAZ2B is chromosome 2 (Ensembl cytogenetic band: 2q24.2), and a representative nucleotide sequence is NM_{—}013450.2 (SEQ ID NO: 59). Although the function of BAZ2B is not known, the bromodomain is a structural motif characteristic of proteins involved in chromatin-dependent regulation of transcription. Bromodomain proteins have been identified as integral components of chromatin remodeling complexes and frequently possess histone acetyltransferase activity. See Jones et al., Genomics 63, 40-45 (2000).

GTPase activating Rap/RanGAP domain-like 1 has also been described as GTPase activating RANGAP domain-like 1 and tuberin-like protein 1. Synonyms for this protein include GARNL1, GRIPE, TULIP1, KIAA0884, DKFZp566D133, and DKFZp667F074. There are at least 2 alternative transcripts for GARNL1 (NP_{—}055805.1 (SEQ ID NO: 60) and NP_{—}919277.2 (SEQ ID NO: 61) encoded by the representative nucleotide sequences NM_{—}014990.1 (SEQ ID NO: 62) and NM_{—}194301.2 (SEQ ID NO: 63). The consensus sequence encodes for a 2036 amino acid protein of 229832 Da. The genomic location of GARNL1 is chromosome 14 (Ensembl cytogenetic band: 14q13.2). GARNL1 interacts with the transcription factor TCF3/isoform E12 in the developing embryonic forebrain, and may be an important transcriptional regulator of downstream target genes under the control of TCF3/E12 by disrupting HLH dimer formation of TCF3/E12 with other proteins. These data suggest that GARNL1 plays a role in neuronal differentiation. See Schwarzbraun et al., Genomics 84, 577-586 (2004); Heng and Tan, J. Biol. Chem. 277, 43152-43159 (2002).

Nucleosome assembly protein 1-like 1 (HSP22-like protein interacting protein; NAP-1 related protein) is also known by the aliases NAP1L1, NRP, NAP1, NAP1L, MGC8688, FLJ16112, and MGC23410. There are at least 2 alternative transcripts for NAP1L1 (NP_{—}004528.1 (SEQ ID NO: 64) and NP_{—}631946.1 (SEQ ID NO: 65) encoded by the representative nucleotide sequences NM_{—}004537.3 (SEQ ID NO: 66) and NM_{—}139207.1 (SEQ ID NO: 67). The consensus sequence encodes for a 391 amino acid protein of 45374 Da. The genomic location of NAP1L1 is chromosome 12 (Ensembl cytogenetic band: 12q21.2). This gene encodes a member of the nucleosome assembly protein (NAP) family. This protein participates in DNA replication and may play a role in modulating chromatin formation and contribute to the regulation of cell proliferation. Alternative splicing of this gene results in several transcript variants; however not all have been fully described. NAP1L1 was also shown to be over-expressed in small-intestinal carcinoid neoplasia. See Eckey et al., Mol. Cell. Biol. 27, 3557-3568 (2007); Rehtanz et al., Mol. Cell. Biol. 24, 2153-2168 (2004); Asahara et al., Mol. Cell. Biol. 22, 2974-2983 (2002); Simon et al., Biochem. 1 297, 389-397 (1994); Kidd et al., Ann. Surg. Oncol. 13, 253-262 (2006).

Synonyms for paraneoplastic neuronal antigen MA1 (neuron- and testis-specific protein 1, and 37 kDa neuronal protein) include PNMA1 and MA1. PNMA1 encodes a 353 amino acid protein of 39761 Da in size (REFSEQ NP_{—}006020.4, SEQ ID NO: 68). The genomic location of BAZ2B is chromosome 14 (Ensembl cytogenetic band: 14q24.3), and a representative nucleotide sequence is NM_{—}006029.4 (SEQ ID NO: 69). PNMA1 was identified as neuronal auto-antigen identified using sera from patients with paraneoplastic neurological syndromes. The function of PNMA1 is not known. See Dalmau et al., Brain 122, 27-39 (1999).

Heterogeneous nuclear ribonucleoprotein A1 (helix-destabilizing protein; single-strand DNA-binding protein UP1; single-strand RNA-binding protein; heterogeneous nuclear ribonucleoprotein A1; heterogeneous nuclear ribonucleoprotein A1B; heterogeneous nuclear ribonucleoprotein B2; hnRNP core protein A1) is also known by the aliases HNRPA1, HNRNPA1, and MGC102835. There are at least 2 alternative transcripts for HNRPA1 (NP_{—}002127.1 (SEQ ID NO: 70) and NP_{—}112420.1 (SEQ ID NO: 71) encoded by the representative nucleotide sequences NM_{—}002136.2 (SEQ ID NO: 72) and NM_{—}031157.2 (SEQ ID NO: 73). The consensus sequence encodes for a 372 amino acid protein of 38846 Da. The genomic location of HNRPA1 is chromosome 12 (Ensembl cytogenetic band: 12q13.13). This gene belongs to the A/B subfamily of ubiquitously expressed heterogeneous nuclear ribonucleoproteins (hnRNPs). The hnRNPs are RNA binding proteins and they complex with heterogeneous nuclear RNA (hnRNA). These proteins are associated with pre-mRNAs in the nucleus and appear to influence pre-mRNA processing and other aspects of mRNA metabolism and transport. While all of the hnRNPs are present in the nucleus, some seem to shuttle between the nucleus and the cytoplasm. The hnRNP proteins have distinct nucleic acid binding properties. The protein encoded by this gene has two repeats of quasi-RRM domains that bind to RNAs. It is one of the most abundant core proteins of hnRNP complexes and it is localized to the nucleoplasm. This protein, along with other hnRNP proteins, is exported from the nucleus, probably bound to mRNA, and is immediately re-imported. Its M9 domain acts as both a nuclear localization and nuclear export signal. The encoded protein is involved in the packaging of pre-mRNA into hnRNP particles, transport of poly A+mRNA from the nucleus to the cytoplasm, and may modulate splice site selection. It is also thought have a primary role in the formation of specific myometrial protein species in parturition. Multiple alternatively spliced transcript variants have been found for this gene but only two transcripts are fully described. These variants have multiple alternative transcription initiation sites and multiple polyA sites. See Lewis et al., Mol. Biol. Cell 18, 1302-1311 (2007); Kim et al., J. Virol. 81, 3852-3865 (2007); Yang et al., Endocrinology 148, 1340-1349 (2007); Hallay et al., J. Biol. Chem. 281, 37159-37174 (2006); Saccone et al., Genomics 12, 171-174 (1992); Ghetti et al., FEBS Lett. 277, 272-276 (1990); Buvoli et al., EMBO 19, 1229-1235 (1990); Biamonti et al., J. Mol. Biol. 207, 491-503 (1989); Buvoli et al., Nucleic Acids Res. 16, 3751-3770 (1988).

Protein tyrosine phosphatase, receptor type, F polypeptide (receptor-linked protein-tyrosine phosphatase LAR; LCA-homolog; leukocyte antigen-related tyrosine phosphatase; leukocyte antigen-related (LAR) PTP receptor) is also known by the aliases PTPRF, EC 3.1.3.48, FLJ43335, FLJ45062, FLJ45567, LAR, LCA-homolog. There are at least 2 alternative transcripts for PTPRF (NP_{—}002831.2 (SEQ ID NO: 74) and NP_{—}569707.2 (SEQ ID NO: 75) encoded by the representative nucleotide sequences NM_{—}002840.3 (SEQ ID NO: 76) and NM_{—}130440.2 (SEQ ID NO: 77). The consensus sequence encodes for a 1897 amino acid protein of 211845 Da. The genomic location of PTPRF is chromosome 1 (Ensembl cytogenetic band: 1p34.2). The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. This PTP possesses an extracellular region, a single transmembrane region, and two tandem intracytoplasmic catalytic domains, and thus represents a receptor-type PTP. The extracellular region contains three Ig-like domains, and 9 non-Ig like domains similar to that of neural-cell adhesion molecule. This PTP was shown to function in the regulation of epithelial cell-cell contacts at adherents junctions, as well as in the control of beta-catenin signaling. An increased expression level of this protein was found in the insulin-responsive tissue of obese, insulin-resistant individuals, and may contribute to the pathogenesis of insulin resistance. Two alternatively spliced transcript variants of this gene, which encode distinct proteins, have been reported. See Hoogenraad et al., Dev. Cell 12, 587-602 (2007); Haapasalo et al., J. Biol. Chem. 282, 9063-9072 (2007); Ruhe et al., Cell. Signal. 18, 1515-1527 (2006); Mander et al., FEBS Lett 579, 3024-3028 (2005); Hashimoto et al., J. Biol. Chem. 267, 13811-13814 (1992); Jirik et al., Cytogenet. Cell Genet. 61, 266-268 (1992); Streuli et al., EMBO J. 9, 2399-2407 (1990); Streuli et al., Proc. Natl. Acad. Sci. U.S.A. 86, 8698-8702 (1989); Streuli et al., J. Exp. Med. 168, 1523-1530 (1988).

Leucine rich repeat (in FLII) interacting protein 1 has also been described as GC-binding factor 2, LRR FLII-interacting protein, leucine-rich repeat flightless-interacting protein 1, TAR RNA-interacting protein, and transcription factor 9-like. Common aliases for this gene include LRRFIP1, FLAP-1, FLIIAP1, GCF-2, HUFI-1, MGC10947, MGC119738, MGC11739, and TRIP. LRRFIP1 encodes a 808 amino acid protein of 39761 Da in size (REFSEQ NP_{—}004726.1, SEQ ID NO: 78). The genomic location of LRRFIP1 is chromosome 2 (Ensembl cytogenetic band: 2q37.3), and a representative nucleotide sequence is NM_{—}004735.2 (SEQ ID NO: 79). This gene functions as a transcriptional repressor which preferentially binds to the GC-rich consensus sequence (5′-AGCCCCCGGCG-3′) and may regulate expression of TNF, EGFR and PDGFA. LRRFIP1 may control smooth muscle cell proliferation following artery injury through PDGFA repression. See Suriano et al., Mol. Cell. Biol. 25, 9073-9081 (2005); Khachigian et al., Circ. Res. 84, 1258-1267 (1999); Fong et al., Genomics 58, 146-157 (1999); Reed et al., J. Biol. Chem. 273, 21594-21602 (1998); Wilson et al., Nucleic Acids Res. 26, 3460-3467 (1998); Liu and Yin, J. Biol. Chem. 273, 7920-7927 (1998).

DKFZP686A01247 is a hypothetical protein identified, in part, by large-scale cDNA sequencing of HeLa cell nuclear phosphoproteins. DKFZP686A01247 encodes a 1083 amino acid protein of 121706 Da in size (REFSEQ NP_{—}055803.1, SEQ ID NO: 80). The genomic location of DKFZP686A01247 is chromosome 4 (Ensembl cytogenetic band: 4p14), and a representative nucleotide sequence is NM_{—}014988.1 (SEQ ID NO: 81). Although the function of this gene is not known, it contains LIM and calponin domains, implicating a role in cytoskeletal organization. See Beausoleil et al., Proc. Natl. Acad. Sci. U.S.A. 101, 12130-12135 (2004); Simpson et al., EMBO Rep. 1, 287-292 (2000).

Proteasome (prosome, macropain) 26S subunit, non-ATPase, 14 has also been described as 26S proteasome non-ATPase regulatory subunit 14, 26S proteasome regulatory subunit rpn11, and 26S proteasome-associated PAD1 homolog. Synonyms for this gene include PSMD14, PAD1, POH1, and rpn11. PSMD14 encodes a 310 amino acid protein of 34577 Da in size (REFSEQ NP_{—}005796.1, SEQ ID NO: 82). The genomic location of PSMD14 is chromosome 2 (Ensembl cytogenetic band: 2q24.2), and a representative nucleotide sequence is NM_{—}005805.3 (SEQ ID NO: 83). PSMD14 is a component of the 26S proteasome, a multiprotein complex that degrades proteins targeted for destruction by the ubiquitin pathway. PSMD14 is also part of a conserved mechanism that determines cellular susceptibility to cytotoxic agents, perhaps by influencing the ubiquitin-dependent proteolysis of transcription factors. See Gallery et al., Mol. Cancer. Ther. 6, 262-268 (2007); Ewing et al., Mol. Syst. Biol. 3, 89 (2007); Nabhan and Ribeiro, J. Biol. Chem. 281 (23), 16099-16107 (2006); Ambroggio et al., PLoS Biol. 2, E2 (2004); Spataro et al., J. Biol. Chem. 272, 30470-30475 (1997).

Additional examples of antigens associated with a likelihood of responsiveness to treatment with proliferation-incompetent tumor cells that express cytokines, e.g., GM-CSF are provided in Table 1 below.

TABLE 1

| Gene | Identity | Genbank # | Location | Size | Function | Notes |
|---|---|---|---|---|---|---|
| RSN | restin (Reed- | NM_198240 | cytoplasm, | 162 Kd | Intermediate filament associated protein that links | |
| | Steinberg cell- | (SEQ ID NO: 84) | cytoskeleton | | endocytic vesicles to microtubules. Restin was | |
| | expressed | | | | found to exhibit proproliferation/survival action on | |
| | intermediate | | | | ovarian cancer cells. Restin is a novel intermediate | |
| | filament- | | | | filament-associated protein highly expressed in the | |
| | associated | | | | Reed-Sternberg cells of Hodgkin's disease. | |
| | protein) (RSN), | | | | | |
| | transcript variant 2 | | | | | |
| DARS2 | aspartyl-tRNA | NM_018122 | cytoplasm, | 74 Kd | Mutations in DARS2, which encodes mitochondrial | |
| | synthetase 2 | (SEQ ID NO: 85) | mitochondrion | | aspartyl-tRNA synthetase, in affected individuals | |
| | (mitochondrial) | | | | with leukoencephalopathy with brain stem and | |
| | (DARS2) | | | | spinal cord involvement and lactate elevation. | |
| | | | | | tRNAs are often overexpressed in cancer cells. | |
| KTN1 | kinectin 1 | NM_182926 | membranes, ER | 156 Kd | Various cellular | Autoantibodies against |
| | (kinesin | (SEQ ID NO: 86) | | | organelles and vesicles | this protein have been |
| | receptor) | | | | are transported along | found in patients with |
| | (KTN1) | | | | the microtubules in the | autoimmune disease. |
| | | | | | cytoplasm. Likewise, | |
| | | | | | membrane recycling of | |
| | | | | | the endoplasmic | |
| | | | | | reticulum (ER), Golgi | |
| | | | | | assembly at the | |
| | | | | | microtubule organizing | |
| | | | | | center, and alignment | |
| | | | | | of lysosomes along | |
| | | | | | microtubules are all | |
| | | | | | related processes. The | |
| | | | | | transport of organelles | |
| | | | | | requires a special class | |
| | | | | | of microtubule- | |
| | | | | | associated proteins | |
| | | | | | (MAPs). One of these | |
| | | | | | is the molecular motor | |
| | | | | | kinesin, an ATPase | |
| | | | | | that moves vesicles | |
| | | | | | unidirectionally toward | |
| | | | | | the plus end of the | |
| | | | | | microtubule. Another | |
| | | | | | such MAP is kinectin, a | |
| | | | | | large integral ER | |
| | | | | | membrane protein. | |
| | | | | | Antibodies directed | |
| | | | | | against kinectin have | |
| | | | | | been shown to inhibit | |
| | | | | | its binding to kinesin. | |
| CENPF | centromere | NM_016343 | cytoplasm, | 368 Kd | This gene encodes a | Autoantibodies against |
| | protein F, | (SEQ ID NO: 87) | chromosome, | | protein that associates | this protein have been |
| | 350/400ka | | nucleus | | with the centromere- | found in patients with |
| | (mitosin) | | | | kinetochore complex. | cancer or graft versus |
| | (CENPF) | | | | The protein is a | host disease. |
| | | | | | component of the | |
| | | | | | nuclear matrix during | |
| | | | | | the G2 phase of | |
| | | | | | interphase. In late G2 | |
| | | | | | the protein associates | |
| | | | | | with the kinetochore | |
| | | | | | and maintains this | |
| | | | | | association through | |
| | | | | | early anaphase. It | |
| | | | | | localizes to the spindle | |
| | | | | | midzone and the | |
| | | | | | intracellular bridge in | |
| | | | | | late anaphase and | |
| | | | | | telophase, respectively, | |
| | | | | | and is thought to be | |
| | | | | | subsequently | |
| | | | | | degraded. The | |
| | | | | | localization of this | |
| | | | | | protein suggests that it | |
| | | | | | may play a role in | |
| | | | | | chromosome | |
| | | | | | segregation during | |
| | | | | | mitotis. It is thought to | |
| | | | | | form either a | |
| | | | | | homodimer or | |
| | | | | | heterodimer. | |
| | | | | | Autoantibodies against | |
| | | | | | this protein have been | |
| | | | | | found in patients with | |
| | | | | | cancer or graft versus | |
| | | | | | host disease. | |
| REST | RE1-silencing | NM_005612 | nucleus | 122 Kd | This gene encodes a | Abnormal expression of |
| | transcription | (SEQ ID NO: 88) | | | transcriptional | REST/NRSF and MYC |
| | factor (REST) | | | | repressor which | in undifferentiated neural |
| | | | | | represses neuronal | stem/progenitor cells |
| | | | | | genes in non-neuronal | causes cerebellum- |
| | | | | | tissues. It is a member | specific tumors. |
| | | | | | of the Kruppel-type zinc | |
| | | | | | finger transcription | |
| | | | | | factor family. It | |
| | | | | | represses transcription | |
| | | | | | by binding a DNA | |
| | | | | | sequence element | |
| | | | | | called the neuron- | |
| | | | | | restrictive silencer | |
| | | | | | element. The protein is | |
| | | | | | also found in | |
| | | | | | undifferentiated | |
| | | | | | neuronal progenitor | |
| | | | | | cells, and it is thought | |
| | | | | | that this repressor may | |
| | | | | | act as a master | |
| | | | | | negative regular of | |
| | | | | | neurogenesis. | |
| | | | | | Alternatively spliced | |
| | | | | | transcript variants have | |
| | | | | | been described; | |
| | | | | | however, their full | |
| | | | | | length nature has not | |
| | | | | | been determined. | |
| UBTF | upstream | NM_014233 | nucleus, | 89 Kd | Upstream binding factor (UBF) is a transcription | |
| | binding | (SEQ ID NO: 89) | nucleolus | | factor required for expression of the 18S, 5.8S, and | |
| | transcription | | | | 28S ribosomal RNAs, along with SL1 (a complex of | |
| | factor, RNA | | | | TBP (MIM 600075) and multiple TBP-associated | |
| | polymerase I | | | | factors or ‘TAFs’). Two UBF polypeptides, of 94 and | |
| | (UBTF) | | | | 97 kD, exist in the human (Bell et al., 1988). UBF is | |
| | | | | | a nucleolar phosphoprotein with both DNA binding | |
| | | | | | and transactivation domains. Sequence-specific | |
| | | | | | DNA binding to the core and upstream control | |
| | | | | | elements of the human rRNA promoter is mediated | |
| | | | | | through several HMG boxes. | |
| SUHW4 | suppressor of | NM_017661 | nucleus | 108 Kd | May be a | |
| | hairy wing | (SEQ ID NO: 90) | | | transcriptional factor. | |
| | homolog 4 | | | | | |
| | (Drosophila) | | | | | |
| | (SUHW4), | | | | | |
| | transcript variant | | | | | |
| BRWD1 | bromodomain | NM_033656 | cytoplasm, | 263 Kd | This gene encodes a member of the WD repeat | |
| | and WD repeat | (SEQ ID NO: 91) | nucleus | | protein family. WD repeats are minimally conserved | |
| | domain | | | | regions of approximately 40 amino acids typically | |
| | containing 1 | | | | bracketed by gly-his and trp-asp (GH-WD), which | |
| | (BRWD1), | | | | may facilitate formation of heterotrimeric or | |
| | transcript variant 2 | | | | multiprotein complexes. Members of this family are | |
| | | | | | involved in a variety of cellular processes, including | |
| | | | | | cell cycle progression, signal transduction, | |
| | | | | | apoptosis, and gene regulation. This protein contains | |
| | | | | | 2 bromodomains and multiple WD repeats, and the | |
| | | | | | function of this protein is not known. This gene is | |
| | | | | | located within the Down syndrome region-2 on | |
| | | | | | chromosome 21. | |
| SDCCAG8 | serologically | NM_006642 | | | | |
| | defined colon | (SEQ ID NO: 92) | | | | |
| | cancer antigen 8 | | | | | |
| | (SDCCAG8) | | | | | |
| SLMAP | sarcolemma | NM_007159 | vesicles, ER, | 95 Kd | Required for protein transport from the ER to the | |
| | associated | (SEQ ID NO: 93) | golgi | | golgi complex. | |
| | protein (SLMAP) | | | | | |
| FAF1 | Fas (TNFRSF6) | NM_131917 | cytoplasm, | 74 Kd | Interaction of Fas | By confocal microscopic |
| | associated | (SEQ ID NO: 94) | nucleus | | ligand (TNFSF6) with | analysis, both Fas and |
| | factor 1 (FAF1) | | | | the FAS antigen | FAF1 were detected in |
| | var 2 | | | | (TNFRSF6) mediates | the cytoplasmic |
| | | | | | programmed cell death, | membrane before Fas |
| | | | | | also called apoptosis, | activation, and in the |
| | | | | | in a number of organ | cytoplasm after Fas |
| | | | | | systems. The protein | activation. FADD and |
| | | | | | encoded by this gene | caspase-8 colocalized |
| | | | | | binds to the | with Fas in Jurkat cells |
| | | | | | cytoplasmic domain of | validating the presence |
| | | | | | FAS and can initiate | of FAF1 in the authentic |
| | | | | | apoptosis or enhance | Fas-DISC. |
| | | | | | apoptosis initiated | Overexpression of FAF1 |
| | | | | | through FAS antigen. | in Jurkat cells caused |
| | | | | | Initiation of apoptosis | significant apoptotic |
| | | | | | by the protein encoded | death. In addition, the |
| | | | | | by this gene requires a | FAF1 deletion mutant |
| | | | | | ubiquitin-like domain | lacking the N terminus |
| | | | | | but not the FAS- | where Fas, FADD, and |
| | | | | | binding domain. | caspase-8 interact |
| | | | | | | protected Jurkat cells |
| | | | | | | from Fas-induced |
| | | | | | | apoptosis demonstrating |
| | | | | | | dominant-negative |
| | | | | | | phenotype. Cell death by |
| | | | | | | overexpression of FAF1 |
| | | | | | | was suppressed |
| | | | | | | significantly in both |
| | | | | | | FADD- and caspase-8- |
| | | | | | | deficient Jurkat cells |
| | | | | | | when compared with that |
| | | | | | | in their parental Jurkat |
| | | | | | | cells. Collectively, our |
| | | | | | | data show that FAF1 is a |
| | | | | | | member of Fas-DISC |
| | | | | | | acting upstream of |
| | | | | | | caspase-8. |
| ETNK1 | ethanolamine | NM_001039481 | cytoplasm | 51 Kd | This gene encodes an ethanolamine kinase, which | |
| | kinase 1 | (SEQ ID NO: 95) | | | functions in the first committed step of the | |
| | (ETNK1), | | | | phosphatidylethanolamine synthesis pathway. This | |
| | transcript variant 2 | | | | cytosolic enzyme is specific for ethanolamine and | |
| | | | | | exhibits negligible kinase activity on choline. | |
| ICA1 | islet cell | NM_004968 | cytoplasm, | 55 Kd | This gene encodes a protein with an arfaptin | |
| | autoantigen 1, | (SEQ ID NO: 96) | golgi, vesicles | (69 Kd | homology domain that is found both in the cytosol | |
| | 69 kDa (ICA1), | | | from NC BI) | and as membrane-bound form on the Golgi complex | |
| | transcript variant 2 | | | | and immature secretory granules. This protein is | |
| | | | | | believed to be an autoantigen in insulin-dependent | |
| | | | | | diabetes mellitus and primary Sjogren's syndrome. | |
| | | | | | Alternatively spliced variants which encode different | |
| | | | | | protein isoforms have been described; however, not | |
| | | | | | all variants have been fully characterized. | |
| ARL6IP5 | ADP- | NM_006407 | cytoplasm, | 22 Kd | Expression of this gene is affected by vitamin A. The | |
| | ribosylation-like | (SEQ ID NO: 97) | ER, | | encoded protein of this gene may be associated with | |
| | factor 6 | | membrane | | the cytoskeleton. A similar protein in rats may play a | |
| | interacting | | | | role in the regulation of cell differentiation. The rat | |
| | protein 5 | | | | protein binds and inhibits the cell membrane | |
| | (ARL6IP5) | | | | glutamate transporter EAAC1. The expression of the | |
| | | | | | rat gene is upregulated by retinoic acid, which | |
| | | | | | results in a specific reduction in EAAC1-mediated | |
| | | | | | glutamate transport. | |
| POLR1B | polymerase | NM_019014 | nucleus | 128 Kd | DNA-dependent RNA | Our analysis indicates |
| | (RNA) I | (SEQ ID NO: 98) | | | polymerase catalyzes | that the ribozymes |
| | polypeptide B, | | | | the transcription of | toward ROCK1 can |
| | 128 kDa | | | | DNA into RNA using | block invasive activity |
| | (POLR1B) | | | | the four ribonucleoside | but not the proliferation |
| | | | | | triphosphates as | of HT1080 cells without |
| | | | | | substrates. RNA | having any effect on |
| | | | | | polymerase i is | expression of ROCK2. |
| | | | | | essentially used to | |
| | | | | | transcribe ribosomal | |
| | | | | | DNA units. | |
| UHMK1 | U2AF homology | NM_175866 | nucleus, | 47 Kd | UHMK1 is a serine threonine kinase nuclear protein | |
| | motif (UHM) | (SEQ ID NO: 99) | cytoplasm | | and is highly expressed in regions of the brain | |
| | kinase 1 | | | | implicated in schizophrenia | |
| | (UHMK1) | | | | | |
| CHMP4C | chromatin | NM_152284 | cytoplasm | 26 Kd | CHMP4C belongs to the chromatin-modifying | |
| | modifying | (SEQ ID NO: 100) | | | protein/charged multivesicular body protein (CHMP) | |
| | protein 4C | | | | family. These proteins are components of ESCRT-III | |
| | (CHMP4C) | | | | (endosomal sorting complex required for transport | |
| | | | | | III), a complex involved in degradation of surface | |
| | | | | | receptor proteins and formation of endocytic | |
| | | | | | multivesicular bodies (MVBs). Some CHMPs have | |
| | | | | | both nuclear and cytoplasmic/vesicular distributions, | |
| | | | | | and one such CHMP, CHMP1A (MIM 164010), is | |
| | | | | | required for both MVB formation and regulation of | |
| | | | | | cell cycle progression. | |
| SPATA5 | spermatogenesis | NM_145207 | cytoplasm | 98 Kd | A novel spermatogenesis associated factor (SPAF) | |
| | associated 5 | (SEQ ID NO: 101) | | | was found to be aberrantly expressed at the malignant | |
| | (SPATA5) | | | | conversion stage in a clonal epidermal model of | |
| | | | | | chemical carcinogenesis. May be involved in FGF | |
| | | | | | signalling - part of the FGF2-NUDT6-SPATA5-SPRY1 | |
| | | | | | locus at human chromosome 4q27-q28.1. | |
| BRAP | BRCA1 | NM_006768 | | | | |
| | associated | (SEQ ID NO: 102) | | | | |
| | protein (BRAP) | | | | | |
| TPR | translocated | NM_003292 | cytoplasm, | 16 Kd | This gene encodes a | PHA665752 is a potent |
| | promoter region | (SEQ ID NO: 103) | nucleus | | large coiled-coil protein | small molecule-selective |
| | (to activated | | | | that forms intranuclear | c-MET inhibitor and is |
| | MET oncogene) | | | | filaments attached to | highly active against |
| | (TPR) | | | | the inner surface of | TPR-MET-transformed |
| | | | | | nuclear pore | cells both biologically |
| | | | | | complexes (NPCs). | and biochemically. |
| | | | | | The protein directly | PHA665752 is also |
| | | | | | interacts with several | active against H441 |
| | | | | | components of the | NSCLC cells. The c- |
| | | | | | NPC. It is required for | MET inhibitor can |
| | | | | | the nuclear export of | cooperate with |
| | | | | | mRNAs and some | rapamycin in therapeutic |
| | | | | | proteins. Oncogenic | inhibition of NSCLC, and |
| | | | | | fusions of the 5′ end of | in vivo studies of this |
| | | | | | this gene with several | combination against c- |
| | | | | | different kinase genes | MET expressing cancers |
| | | | | | occur in some | would be merited. |
| | | | | | neoplasias. | |
| PSMD7 | proteasome | NM_002811 | | | | |
| | (prosome, | (SEQ ID NO: 104) | | | | |
| | macropain) 26S | | | | | |
| | subunit, non- | | | | | |
| | ATPase, 7 | | | | | |
| | (Mov34 | | | | | |
| | homolog) | | | | | |
| | (PSMD7) | | | | | |
| NRBF2 | nuclear receptor | NM_030759 | cytoplasm, | 32 Kd | A protein named nuclear receptor binding factor-2 | |
| | binding factor 2 | (SEQ ID NO: 105) | nucleus | | (NRBF-2) was identified by yeast two-hybrid screening, | |
| | (NRBF2) | | | | as an interaction partner of peroxisome proliferator- | |
| | | | | | activated receptor alpha as well as several other | |
| | | | | | nuclear receptors. NRBF-2 exhibited a gene activation | |
| | | | | | function, when tethered to a heterologous DNA binding | |
| | | | | | domain, in both mammalian cells and yeast. The | |
| | | | | | activation domain, their small size (COPR1, 26.9 kDa; | |
| | | | | | COPR2, 32.4 kDa), and strict dependence on AF-2 for | |
| | | | | | interaction distinguish COPR1 and COPR2 from the | |
| | | | | | SMRT/NCoR type of corepressor and may dampen | |
| | | | | | rather than repress NR-mediated gene expression. | |
| ROCK2 | Rho-associated, | NM_004850 | cytoplasm, | 161 Kd | The protein encoded by this gene is a serine/threonine | |
| | coiled-coil | (SEQ ID NO: 106) | nucleus | | kinase that regulates cytokinesis, smooth muscle | |
| | containing | | | | contraction, the formation of actin stress fibers and | |
| | protein kinase 2 | | | | focal adhesions, and the activation of the c-fos serum | |
| | (ROCK2) | | | | response element. This protein, which is an isozyme of | |
| | | | | | ROCK1 is a target for the small GTPase Rho. | |
| MAML3 | mastermind-like | NM_018717 | nucleus | 122 Kd | Lin et al. (2002) found | Notch has also been |
| | 3 (Drosophila) | (SEQ ID NO: 107) | | | that the MAML proteins | linked to the |
| | (MAML3) | | | | stabilized and | pathogenesis of small- |
| | | | | | participated in the | cell lung |
| | | | | | Notch/CSL DNA- | cancer (SCLC), a tumor |
| | | | | | binding complex and | with neuroendocrine |
| | | | | | enhanced the | (NE) differentiation. |
| | | | | | activation of | |
| | | | | | transcription from the | |
| | | | | | target promoter. They | |
| | | | | | found that activation of | |
| | | | | | the target promoter by | |
| | | | | | NOTCH3 (600276) and | |
| | | | | | NOTCH4 (164951) was | |
| | | | | | more efficiently | |
| | | | | | potentiated by MAML3 | |
| | | | | | than by MAML1 or | |
| | | | | | MAML2. | |
| FAM50A | family with | NM_004699 | nucleus | 40 Kd | May be a DNA-binding protein or transcriptional factor. | |
| | sequence | (SEQ ID NO: 108) | | | | |
| | similarity 50, | | | | | |
| | member A | | | | | |
| | (FAM50A) | | | | | |
| STK39 | serine threonine | NM_013233 | cytoplasm, | 60 Kd | This gene encodes a | Lower mRNA expression |
| | kinase 39 | (SEQ ID NO: 109) | nucleus | | serine/threonine kinase | of HERPUD1, STK39, |
| | (STE20/SPS1 | | | | that is thought to | DHCR24, and SOCS2 in |
| | homolog, yeast) | | | | function in the cellular | primary prostate tumors |
| | (STK39) | | | | stress response | was correlated with a |
| | | | | | pathway. The kinase is | higher incidence of |
| | | | | | activated in response | metastases after radical |
| | | | | | to hypotonic stress, | prostatectomy. |
| | | | | | leading to | Tolerance induction by |
| | | | | | phosphorylation of | mixed chimerism and |
| | | | | | several cation-chloride- | costimulation blockade is |
| | | | | | coupled cotransporters. | a promising approach to |
| | | | | | The catalytically active | avoid |
| | | | | | kinase specifically | immunosuppression, but |
| | | | | | activates the p38 MAP | the molecular basis of |
| | | | | | kinase pathway, and its | tolerant T lymphocytes |
| | | | | | interaction with p38 | remains elusive. The |
| | | | | | decreases upon | genome-wide gene |
| | | | | | cellular stress, | expression profile of |
| | | | | | suggesting that this | murine T lymphocytes |
| | | | | | kinase may serve as an | after tolerance induction |
| | | | | | intermediate in the | by allogeneic bone |
| | | | | | response to cellular | marrow transplantation |
| | | | | | stress. | (BMT) and costimulatory |
| | | | | | | blockade using the anti- |
| | | | | | | CD40L antibody MR1 |
| | | | | | | has been investigated. |
| | | | | | | Molecular functions, |
| | | | | | | biological processes, |
| | | | | | | cellular locations, and |
| | | | | | | coregulation of identified |
| | | | | | | genes were determined. |
| | | | | | | A total of 113 unique |
| | | | | | | genes exhibited a |
| | | | | | | significant differential |
| | | | | | | expression between the |
| | | | | | | lymphocytes of MR1- |
| | | | | | | treated Tolerance (TOL) |
| | | | | | | and untreated recipients |
| | | | | | | Control (CTRL). The |
| | | | | | | majority of genes |
| | | | | | | upregulated in the TOL |
| | | | | | | group are involved in |
| | | | | | | several signal |
| | | | | | | transduction cascades |
| | | | | | | such as members of the |
| | | | | | | MAPKKK cascade (IL6, |
| | | | | | | Tob2, Stk39, and D |

| | | | | | |
|---|---|---|---|---|---|
| TRMT5 | TRM5 tRNA | NM_020810 | nucleus, | 58 Kd | tRNAs contain as many as 13 or 14 nucleotides that |
| | methyltransferase | (SEQ ID NO: 110) | cytoplasm | | are modified posttranscriptionally by enzymes that are |
| | 5 homolog | | | | highly specific for particular nucleotides in the tRNA |
| | (TRMT5) | | | | structure. TRMT5 methylates the N1 position of |
| | | | | | guanosine-37 (G37) in selected tRNAs using S- |
| | | | | | adenosyl methionine. |
| RPIA | ribose 5- | NM_144563 | cytoplasm | 33 Kd | This enzyme belongs to the family of isomerases, |
| | phosphate | (SEQ ID NO: 111) | | | specifically those intramolecular oxidoreductases |
| | epimerase | | | | interconverting aldoses and ketoses. The systematic |
| | (RPIA) | | | | name of this enzyme class is D-ribose-5-phosphate |
| | | | | | aldose-ketose-isomerase. Other names in common use |
| | | | | | include phosphopentosisomerase, |
| | | | | | phosphoriboisomerase, ribose phosphate isomerase, |
| | | | | | 5-phosphoribose isomerase, D-ribose 5-phosphate |
| | | | | | isomerase, and D-ribose-5-phosphate ketol-isomerase. |
| | | | | | This enzyme participates in pentose phosphate |
| | | | | | pathway and carbon fixation. |

A summary of antigens associated with a likelihood of responsiveness to treatment with proliferation-incompetent tumor cells that express cytokines, e.g., GM-CSF are provided in Tables 2, 3 and 4 in the Examples below.

### 4.3 METHODS OF USING ANTIGENS

The antigens provided herein find use in a variety of methods, including methods for determining whether an immune response against cancer cells has been induced in a subject, methods for determining whether an immune response effective to treat, prevent, or ameliorate a symptom of lung cancer in a subject has been induced in the subject, methods for determining whether a subject afflicted with lung cancer is likely to respond to treatment with genetically modified tumor cells that produce GM-CSF, and methods for assessing the effectiveness of lung cancer therapy with genetically modified tumor cells that express GM-CSF to treat or ameliorate a symptom of lung cancer of a subject in need thereof. In certain embodiments, the lung cancer is non-small cell lung cancer (NSCLC).

In another aspect, provided herein is a method for determining whether an immune response effective to treat, prevent, or ameliorate a symptom of non-small cell lung cancer in a subject has been induced in the subject, comprising detecting an immune response against an antigen listed in Table 2, 3 or 4, wherein detecting said antigen indicates that an immune response effective to treat, prevent, or ameliorate a symptom of non-small cell lung cancer has been induced in the subject. In certain embodiments, an immune response is detected against an antigen identified in Table 2. In certain embodiments, an immune response is detected against an antigen identified in Table 3. In certain embodiments, an immune response is detected against an antigen identified in Table 4. In certain embodiments, an immune response against 1, 2, 3, 4, 5, 6, 7, 8, 9, 10, 11, 12, 13, 14, 15, 16, 17, 18, 19, 20, 21, 22, 23, 24, 25 or more of the antigens identified in Table 2 is detected. In certain embodiments, an immune response against 1, 2, 3, 4, 5, 6, 7, 8, 9, 10, 11, 12, 13, 14, 15, 16, 17, 18, 19, 20, 21, 22, 23, 24, 25 or more of the antigens identified in Table 3 is detected. In certain embodiments, an immune response against 1, 2, 3, 4, 5, 6, 7, 8, 9, 10, 11, 12, 13, 14, 15, 16, 17, 18, 19, 20, 21, 22, 23, 24, 25 or more of the antigens identified in Table 4 is detected.

In certain embodiments, the immune response that has been induced is effective to prevent lung cancer in the subject. In certain embodiments, the immune response that has been induced is effective to treat lung cancer in the subject. In certain embodiments, the immune response that has been induced is effective to ameliorate a symptom of lung cancer in the subject. In certain embodiments, the symptom of lung cancer that is ameliorated is selected from the group consisting of cancer-associated pain and metastasis. In certain embodiments, the immune response is effective to result in decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, or increased impact on circulating T cell and dendritic cell numbers, phenotype, and function, cytokine response, reduced metastasis as measured by bone scan/MRI or other methods, increased time to progression, decreased serum concentrations of ICTP, decreased concentrations of serum C-reactive protein or decreased numbers of circulating tumor cells (CTCs).

Any method known by those skilled in the art for detecting an immune response can be used in accordance with the methods provided herein. In certain embodiments, the immune response is detected by western blot. In certain embodiments, the immune response is detected by ELISA. In certain embodiments, the immune response is detected by protein array analysis.

### 4.4 CORRELATION OF IMMUNE RESPONSE WITH LIKELIHOOD OF RESPONDING OR RESPONSIVENESS

Clinical datasets of immune responses with clinical outcome data can be used to correlate immune responses with likelihood of responding to cancer therapy or with responsiveness to cancer therapy.

Any method known in the art, without limitation, can be used to assess the immune response of a subject administered a cancer therapy, e.g., a cell-based cancer immunotherapy such as, e.g., GVAX® therapy. For example, such immune responses can be assessed by western blot, by ELISA, by protein array analysis, and the like.

Similarly, any method known in the art can be used to determine whether an immune response is correlated with responsiveness to cancer therapy. Typically, P values are used to determine the statistical significance of the correlation, such that the smaller the P value, the more significant the measurement. Preferably the P values will be less than 0.05 (or 5%). More preferably, P values will be less than 0.01. P values can be calculated by any means known to one of skill in the art. For the purposes of correlating an immune response with responsiveness to cancer therapy, P values can be calculated using Fisher's Exact Test. See, e.g., David Freedman, Robert Pisani & Roger Purves, 1980, STATISTICS, W. W. Norton, New York. P values may be calculated using Student's paired and/or unpaired t-test and the non-parametric Kruskal-Wallis test (Statview 5.0 software, SAS, Cary, N.C.).

Typically, immune responses are measured from biological samples obtained from a subject. Biological samples from a subject include, for example and without limitation, blood, blood plasma, serum, urine, saliva, tissue swab and the like.

### 4.5 CONSTRUCTING AN ALGORITHM

In one aspect, provided herein is a method of constructing an algorithm that correlates immune response data with responsiveness to cancer therapy, e.g., a cell-based cancer immunotherapy such as, e.g., GVAX® therapy. In one embodiment, the method of constructing the algorithm comprises creating a rule or rules that correlate immune response data with responsiveness to cancer therapy, e.g., a cell-based cancer immunotherapy such as, e.g., GVAX® therapy.

In one embodiment, a data set comprising immune response data and clinical outcome data about each subject in a set of subjects is assembled. Any method known in the art can be used to collect immune response data. Examples of methods of collecting such data are provided above. Any method known in the art can be used for collecting clinical outcome data.

In some embodiments, the data set comprises immune responses against one or more antigens as described herein. In some embodiments, the data set comprises immune responses against 1, 2, 3, 4, 5, 6, 7, 8, 9, 10, 11, 12, 13, 14, 15, 16, 17, 18, 19, 20, 21, 22, 23, 24, 25, or more antigens.

In some embodiments, the clinical outcome data comprises information regarding cancer-associated pain and/or metastasis. In some embodiments, the clinical outcome data comprises information regarding the serum concentrations of tumor specific markers, overall survival time, progression-free survival, tumor size, metastasis marker response, impact on minimal residual disease, induction of antibody response to the cancer cells that have been rendered proliferation-incompetent, induction of delayed-type-hypersensitivity (DTH) response to injections of autologous tumor, induction of T cell response to autologous tumor or candidate tumor-associated antigens, and/or impact on circulating T cell and dendritic cell numbers, phenotype, and/or function, cytokine response and decrease in number of circulating tumor cells (CTC).

The immune response and clinical outcome data in the data set can be represented or organized in any way known in the art. In one embodiment, the data are displayed in the form of a graph. In another embodiment, the immune response and clinical outcome data in the data set are displayed in the form of a chart.

In one aspect, an algorithm is formulated that correlates the immune response with the clinical outcome data in the data set. In one embodiment, a clinical outcome cutoff point is defined. In some embodiments, the clinical outcome cutoff point is determined relative to a reference subject, and the cutoff point is the value above or below which a subject is defined as responsive to the cancer therapy and below or above which a subject is defined nonresponsive to the cancer therapy. One skilled in the art will recognize that for some clinical indicators, e.g., survival time, an increase in the clinical indicator indicates responsiveness, while for other clinical indicators, e.g., tumor size or tumor marker, an increase in the clinical indicator indicates nonresponsiveness.

In another embodiment, the upper or lower clinical cutoff point is used to define the level of immune responsiveness. In one embodiment, the number of antigens against which an immune response and/or the concentration of antibodies against an antigen against which an immune response is raised is correlated with the clinical outcome data. An immune response cutoff point can be selected such that most subjects having an immune response against more than that number of antigens or with a concentration of antibodies higher than the cutoff concentration in the data set are immunologically responsive to treatment (IR-R), and most subjects having fewer or less than that number are immunologically not responsive (IR-N). By definition, a subject in the data set with clinical outcome data more or less than, as appropriate, the clinical outcome cutoff is clinically responsive (“CL-R”) to the cancer treatment, and a subject in the data set with fewer or more than, as appropriate, the clinical outcome cutoff is clinically nonresponsive (“CL-N”) to the treatment. Thus, in one embodiment, a immune response cutoff point is selected that produces the greatest percentage of subject in the data set that are either clinically and immunologically responsive (“IR-R, CL-R”), or immunologically responsive and clinically nonresponsive (“IR-N, CL-N”).

While this simple algorithm can provide a useful approximation of the relationship between the immune response and clinical outcome data in the data set, in most cases there will be a significant number of subjects that are clinically nonresponsive but immunologically responsive (“CL-N, IR-R”), or immunologically nonresponsive but clinically responsive (“CL-R, IR-N”). These discordant results are a measure of the inaccuracy of the algorithm. Thus, in some embodiments, the algorithm is further modified to reduce the percentage of discordant results in the data set.

In another embodiment, the percentage of discordant results is reduced by assigning differential weight values to immune responses against one or more antigens observed in the data set. An algorithm that does not include this step assumes that each immune response in the data set contributes equally to the overall clinical outcome. In many cases this will not be true. For example, there may be a antigen in a data set that is almost always correlated with responsiveness to a cancer treatment. That is, almost every subject that has an immune response against the antigen is clinically responsive, even those subjects having an immune response against only one or two total antigens. In one embodiment, immune responses against such antigens are “weighted,” e.g., assigned an increased score. An immune response can be assigned a weight of, for example, two, three, four, five, six, seven, eight or more. For example, an immune response assigned a weight of 2 can be counted as two immune responses in a subject. Fractional weighting values can also be assigned. In certain embodiments, a value between zero and one can be assigned when an immune response is weakly associated with a clinical outcome. In another embodiment, values of less than zero can be assigned, wherein an immune response is associated with an negative clinical outcome to the anti-viral treatment.

One of skill in the art will appreciate that there is a tradeoff involved in assigning an increased weight to certain immune responses. As the weight of the immune response is increased, the number of IR-R, CL-N discordant results may increase. Thus, assigning a weight to an immune response that is too great may increase the overall discordance of the algorithm. Accordingly, in one embodiment, a weight is assigned to an immune response that balances the reduction in IR-N, CL-R results with the increase in IR-R, CL-N results.

In another embodiment, the interaction of different immune responses in the data set with each other is also factored into the algorithm. For example, it might be found that two or more immune responses behave synergistically, i.e., that the coincidence of the immune responses in a subject contributes more significantly to the clinical outcome than would be predicted based on the effect of each immune response independent of the other. Alternatively, it might be found that the coincidence of two or more immune responses in a subject contributes less significantly to the clinical outcome than would be expected from the contributions made to resistance by each immune response when it occurs independently. Also, two or more immune responses may be found to occur more frequently together than as independent immune responses. Thus, in one embodiment, immune responses occurring together are weighted together. For example, only one of the immune responses is assigned a weight of 1 or greater, and the other immune response or immune responses are assigned a weight of zero, in order to avoid an increase in the number of IR-R, CL-N discordant results.

In another aspect, the immune response cutoff point can be used to define a clinical outcome cutoff point by correlating the concentrations of antibody induced as well as the antigens against which immune responses are induced in the data set with the clinical outcome.

In one embodiment, an algorithm is constructed that factors in the requirement for a certain concentration of antibody that is induced

By using, for example, the methods discussed above, the algorithm can be designed to achieve any desired result. In one embodiment, the algorithm is designed to maximize the overall concordance (the sum of the percentages of the IR-R, CL-R and the IR-N, CL-N groups, or 100−(percentage of the IR-N, CL-R+IR-R, CL-N groups). In some embodiments, the overall concordance is greater than 75%, 80%, 85%, 90% or 95%. In one embodiment, the algorithm is designed to minimize the percentage of IR-R, CL-N results. In another embodiment, the algorithm is designed to minimize the percentage of IR-N, CL-R results. In another embodiment, the algorithm is designed to maximize the percentage of IR-R, CL-R results. In another embodiment, the algorithm is designed to maximize the percentage of IR-N, CL-N results.

At any point during the construction of the algorithm, or after it is constructed, it can be further tested on a second data set. In one embodiment, the second data set consists of subjects that are not included in the data set, i.e., the second data set is a naïve data set. In another embodiment, the second data set contains one or more subjects that were in the data set and one or more subjects that were not in the data set. Use of the algorithm on a second data set, particularly a naïve data set, allows the predictive capability of the algorithm to be assessed. Thus, in one embodiment, the accuracy of an algorithm is assessed using a second data set, and the rules of the algorithm are modified as described above to improve its accuracy. In another embodiment, an iterative approach is used to create the algorithm, whereby an algorithm is tested and then modified repeatedly until a desired level of accuracy is achieved.

### 4.6 USING AN ALGORITHM TO PREDICT THE RESPONSIVENESS OF A SUBJECT

In another aspect, also provided herein is a method for using an algorithm to predict the responsiveness of a subject to a cancer therapy based on the immune responses of the subject. In one embodiment, the method comprises detecting, in the subject or derivative of the subject, the presence or absence of an immune response against one or more antigens associated with responsiveness to a cancer therapy, applying the rules of the algorithm to the detected immune responses, wherein a subject that satisfies the rules of the algorithm is responsive or partially responsive to the treatment, and a subject that does not satisfy the rules of the algorithm is nonresponsive to the treatment.

In another embodiment, the method comprises detecting, in the subject or derivative of the subject, the presence or absence of an immune response against one or more antigens associated with responsiveness to a cancer therapy, applying the rules of the algorithm to the detected mutations, wherein a score equal to, or greater than the immune response cutoff score indicates that the subject is responsive or partially responsive to the treatment, and a score less than the immune response cutoff score indicates that the subject is nonresponsive to the treatment.

In yet another embodiment, the method comprises detecting, in the subject or derivative of the subject, the presence or absence of an immune response against one or more antigens associated with responsiveness to a cancer therapy, applying the rules of the algorithm to the detected immune responses, wherein a score less than zero indicates that the subject is not likely to respond to the cancer treatment.

### 4.7 IMMUNOGENIC COMPOSITIONS COMPRISING CELLS EXPRESSING CYTOKINES

The methods provided herein relate, in part, to methods relating to the effectiveness of cancer therapy with cells genetically altered to express cytokines, e.g., GM-CSF. Cancer therapies with cells genetically altered to express cytokines are extensively described hereinafter.

In one aspect, the method of treating lung cancer in a subject comprises administering genetically modified cytokine-expressing cells to the subject as part of a therapeutic treatment for cancer. The method can be carried out by genetically modifying (transducing) a first population of tumor cells to produce a cytokine, e.g., GM-CSF, and administering the first population of tumor cells alone or in combination with a second population of tumor cells to the subject. The tumor cells may be tumor cells from the same individual (autologous), from a different individual (allogeneic) or bystander cells (further described below). The tumor cells may be from a tumor cell line of the same type as the tumor or cancer being treated, e.g., the modified cells are lung cells or lung cancer cells and the patient has lung cancer. Alternatively, the tumor cells may be from a tumor cell line of a different type as the tumor or cancer being treated, e.g., the modified cells are prostate cells or prostate cancer cells and the patient has lung cancer.

Typically the genetically modified tumor cells are rendered proliferation incompetent prior to administration. In one embodiment, the mammal is a human who harbors lung tumor cells of the same type as the genetically modified cytokine-expressing tumor cells. In a preferred embodiment, an improved therapeutic outcome is evident following administration of the genetically modified cytokine-expressing tumor cells to the subject. Any of the various parameters of an improved therapeutic outcome for a non-small cell lung cancer patient known to those of skill in the art may be used to assess the efficacy of genetically modified cytokine-expressing tumor cell therapy.

In still another aspect, the method is effective to stimulate a systemic immune response in a lung cancer patient, comprising administering a therapeutically effective amount of proliferation incompetent genetically modified cytokine-expressing cells to the subject. The systemic immune response to the cytokine-expressing cells may result in regression or inhibition of the growth of lung tumor cells. In certain embodiments, the lung cancer is non-small cell lung cancer (NSCLC). In some embodiments, the non-small cell lung cancer is early stage non-small cell lung cancer. In some embodiments, the non-small cell lung cancer is advanced stage non-small cell lung cancer. In some embodiments, non-small cell lung cancer is stage IIIA, IIIB, or IV non-small cell lung cancer.

In some embodiments, the primary lung tumor has been treated, e.g., by ablation or rescission and metastases of the primary lung cancer are treated by immunotherapy as described herein.

In one preferred embodiment, a viral or nonviral vector is utilized to deliver a human GM-CSF transgene (coding sequence) to a human tumor cell ex vivo. After transduction, the cells are irradiated to render them proliferation incompetent. The proliferation incompetent GM-CSF expressing tumor cells are then re-administered to the patient (e.g., by the intradermal or subcutaneous route) and thereby function as a cancer immunotherapy. The human tumor cell may be a primary tumor cell or derived from a tumor cell line.

In general, the genetically modified tumor cells include one or more of autologous tumor cells, allogeneic tumor cells and tumor cell lines (i.e., bystander cells). The tumor cells may be transduced in vitro, ex vivo or in vivo. Autologous and allogeneic cancer cells that have been genetically modified to express a cytokine, e.g., GM-CSF, followed by readministration to a patient for the treatment of cancer are described in U.S. Pat. Nos. 5,637,483, 5,904,920 and 6,350,445, expressly incorporated by reference herein. A form of GM-CSF-expressing genetically modified tumor cells or a “cytokine-expressing cellular immunotherapy” (“GVAX”®), for the treatment of pancreatic cancer is described in U.S. Pat. Nos. 6,033,674 and 5,985,290, expressly incorporated by reference herein. A universal immunomodulatory genetically modified bystander cell line is described in U.S. Pat. No. 6,464,973, expressly incorporated by reference herein.

An allogeneic form of GVAX® wherein the cellular immunotherapy comprises one or more prostate tumor cell lines selected from the group consisting of DU145, PC-3, and LNCaP is described in WO/0026676, expressly incorporated by reference herein. LNCaP is a PSA-producing prostate tumor cell line, while PC-3 and DU-145 are non-PSA-producing prostate tumor cell lines (Pang S. et al., Hum Gene Ther. 1995 November; 6(11):1417-1426).

Clinical trials employing GM-CSF-expressing cellular immunotherapy (GVAX®) have been undertaken for treatment of prostate cancer, melanoma, lung cancer, pancreatic cancer, renal cancer, and multiple myeloma. A number of clinical trials using GVAX® cellular immunotherapy have been described, most notably in melanoma, and prostate, renal and pancreatic carcinoma (Simons J W et al. Cancer Res. 1999; 59:5160-5168; Simons J W et al., Cancer Res 1997; 57:1537-1546; Soiffer R et al. Proc. Natl. Acad. Sci. USA 1998; 95:13141-13146; Jaffee, et al. J Clin Oncol 2001; 19:145-156; Salgia et al. J Clin Oncol 2003 21:624-30; Soiffer et al. J Clin Oncol 2003 21:3343-50; Nemunaitis et al. J Natl Cancer Inst. 2004 Feb. 18 96(4):326-31).

By way of example, in one approach, genetically modified GM-CSF expressing tumor cells are provided as an allogeneic or bystander cell line and one or more additional cancer therapeutic agents is included in the treatment regimen. In another approach, one or more additional transgenes are expressed by an allogeneic or bystander cell line while a cytokine (i.e., GM-CSF) is expressed by autologous or allogeneic cells. The GM-CSF coding sequence is introduced into the tumor cells using a viral or non-viral vector and routine methods commonly employed by those of skill in the art. The preferred coding sequence for GM-CSF is the genomic sequence described in Huebner K. et al., Science 230(4731):1282-5,1985, however, in some cases the cDNA form of GM-CSF finds utility in practicing the methods (Cantrell et al., Proc. Natl. Acad. Sci., 82, 6250-6254, 1985).

The genetically modified tumor cells can be cryopreserved prior to administration. Preferably, the genetically modified tumor cells are irradiated at a dose of from about 50 to about 200 rads/min, even more preferably, from about 120 to about 140 rads/min prior to administration to the patient. Preferably, the cells are irradiated with a total dose sufficient to inhibit substantially 100% of the cells from further proliferation. Thus, desirably the cells are irradiated with a total dose of from about 10,000 to 20,000 rads, optimally, with about 15,000 rads. Typically more than one administration of cytokine (e.g., GM-CSF) producing cells is delivered to the subject in a course of treatment. Dependent upon the particular course of treatment, multiple injections may be given at a single time point with the treatment repeated at various time intervals. For example, an initial or “priming” treatment may be followed by one or more “booster” treatments. Such “priming” and “booster” treatments are typically delivered by the same route of administration and/or at about the same site. When multiple doses are administered, the first immunization dose may be higher than subsequent immunization doses. For example, a 5×10⁶ prime dose may be followed by several booster doses of 10⁶ to 3×10⁶ GM-CSF producing cells.

A single injection of cytokine-producing cells is typically between about 10⁶ to 10⁸ cells, e.g., 1×10⁶, 2×10⁶, 3×10⁶, 4×10⁶, 5×10⁶, 6×10⁶, 7×10⁶, 8×10⁶, 9×10⁶, 10⁷, 2×10⁷, 5×10⁷, or as many as 10⁸ cells. In one embodiment, there are between 10⁶ and 10⁸ cytokine-producing cells per unit dose. The number of cytokine-producing cells may be adjusted according, for example, to the level of cytokine produced by a given cytokine producing cellular immunotherapy.

In some embodiments, cytokine-producing cells are administered in a dose that is capable of producing at least 500 ng of GM-CSF per 24 hours per one million cells. Determination of optimal cell dosage and ratios is a matter of routine determination and within the skill of a practitioner of ordinary skill, in light of the disclosure provided herein.

In treating a lung cancer patient according to the methods described herein, the attending physician may administer lower doses of the cytokine-expressing tumor cell immunotherapy and observe the patient's response. Larger doses of the cytokine-expressing tumor cell immunotherapy may be administered until an improved therapeutic outcome is evident.

Cytokine-producing cells described herein are processed to remove most of the additional components used in preparing the cells. In particular, fetal calf serum, bovine serum components, or other biological supplements in the culture medium are removed. In one embodiment, the cells are washed, such as by repeated gentle centrifugation, into a suitable pharmacologically compatible excipient. Compatible excipients include various cell culture media, isotonic saline, with or without a physiologically compatible buffer, for example, phosphate or hepes, and nutrients such as dextrose, physiologically compatible ions, or amino acids, particularly those devoid of other immunogenic components. Carrying reagents, such as albumin and blood plasma fractions and inactive thickening agents, may also be used.

4.7.1. Autologous Cells

The use of autologous genetically modified GM-CSF expressing cells provides advantages since each patient's tumor expresses a unique set of tumor antigens that can differ from those found on histologically-similar, MHC-matched tumor cells from another patient. See, e.g., Kawakami et al., J. Immunol., 148, 638-643 (1992); Darrow et al., J. Immunol., 142, 3329-3335 (1989); and Horn et al., J. Immunother., 10, 153-164 (1991).

In one preferred aspect, the method of treating lung cancer comprises: (a) obtaining tumor cells from a mammalian subject harboring a lung tumor; (b) genetically modifying the tumor cells to render them capable of producing an increased level of GM-CSF relative to unmodified tumor cells; (c) rendering the modified tumor cells proliferation incompetent; and (d) readministering the genetically modified tumor cells to the mammalian subject from which the tumor cells were obtained or to a mammal with the same MHC type as the mammal from which the tumor cells were obtained. The administered tumor cells are autologous and MHC-matched to the host. Preferably, the composition is administered intradermally, subcutaneously or intratumorally to the mammalian subject.

In some cases, a single autologous tumor cell may express GM-CSF alone or GM-CSF plus one or more additional transgenes. In other cases, GM-CSF and the one or more additional transgenes may be expressed by different autologous tumor cells. In one aspect of the methods provided herein, an autologous tumor cell is modified by introduction of a vector comprising a nucleic acid sequence encoding GM-CSF, operatively linked to a promoter and expression/control sequences necessary for expression thereof. In another aspect, the same autologous tumor cell or a second autologous tumor cell can be modified by introduction of a vector comprising a nucleic acid sequence encoding at least one additional transgene operatively linked to a promoter and expression/control sequences necessary for expression thereof. The nucleic acid sequence encoding the one or more transgenes can be introduced into the same or a different autologous tumor cell using the same or a different vector. The nucleic acid sequence encoding the transgene(s) may or may not further comprise a selectable marker sequence operatively linked to a promoter. Desirably, the autologous tumor cell expresses high levels of GM-CSF.

4.7.2. Allogeneic Cells

Researchers have sought alternatives to autologous and MHC-matched cells as tumor immunotherapy, as reviewed by Jaffee et al., Seminars in Oncology, 22, 81-91 (1995). Early tumor immunotherapy strategies were based on the understanding that the vaccinating cells function as the antigen presenting cells (APCs) that present tumor antigens on their MHC class I and II molecules, and directly activate the T cell arm of the immune system. The results of Huang et al. (Science, 264, 961-965, 1994), indicate that professional APCs of the host rather than the vaccinating cells prime the T cell arm of the immune system by secreting cytokine(s) such as GM-CSF such that bone marrow-derived APCs are recruited to the region of the tumor. The bone marrow-derived APCs take up the whole cellular protein of the tumor for processing, and then present the antigenic peptide(s) on their MHC class I and II molecules, thereby priming both the CD4+ and the CD8+ T cell arms of the immune system, resulting in a systemic tumor-specific anti-tumor immune response. Without being bound by theory, these results suggest that it may not be necessary or optimal to use autologous or MHC-matched cells in order to elicit an anti-cancer immune response and that the transfer of allogeneic MHC genes (from a genetically dissimilar individual of the same species) can enhance tumor immunogenicity. More specifically, in certain cases, the rejection of tumors expressing allogeneic MHC class I molecules has resulted in enhanced systemic immune responses against subsequent challenge with the unmodified parental tumor. See, e.g., Jaffee et al., supra, and Huang et al., supra.

As used herein, a “tumor cell line” comprises cells that were initially derived from a tumor. Such cells typically exhibit indefinite growth in culture. In one aspect, the method for treating lung cancer comprises: (a) obtaining a tumor cell line; (b) genetically modifying the tumor cell line to render the cells capable of producing an increased level of a cytokine, e.g., GM-CSF, relative to the unmodified tumor cell line; (c) rendering the modified tumor cell line proliferation incompetent; and (d) administering the tumor cell line to a mammalian subject (host) having at least one tumor that is of the same type of tumor as that from which the tumor cell line was obtained. In some embodiments, the administered tumor cell line is allogeneic and is not MHC-matched to the host. Such allogeneic lines provide the advantage that they can be prepared in advance, characterized, aliquoted in vials containing known numbers of transgene (e.g., GM-CSF) expressing cells and stored (i.e. frozen) such that well characterized cells are available for administration to the patient. Methods for the production of genetically modified allogeneic cells are described for example in WO 00/72686, expressly incorporated by reference herein.

In one approach to preparing genetically modified GM-CSF expressing allogeneic cells, a nucleic acid sequence (transgene) encoding GM-CSF alone or in combination with the nucleic acid coding sequence for one or more additional transgenes is introduced into a cell line that is an allogeneic tumor cell line (i.e., derived from an individual other than the individual being treated). In another approach, a nucleic acid sequence (transgene) encoding GM-CSF alone or in combination with the nucleic acid coding sequence for one or more additional transgenes is introduced into separate allogeneic tumor cell lines. In yet another approach two or more different genetically modified allogeneic GM-CSF expressing cell lines (e.g. LNCAP and PC-3) are administered in combination, typically at a ratio of 1:1. In general, the cell or population of cells is from a tumor cell line of the same type as the tumor or cancer being treated, e.g. lung cancer. However, the cell or population of cells may be from a tumor cell line of a different type compared to the tumor or cancer being treated. The nucleic acid sequence encoding the transgene(s) may be introduced into the same or a different allogeneic tumor cell using the same or a different vector. The nucleic acid sequence encoding the transgene(s) may or may not further comprise a selectable marker sequence operatively linked to a promoter. Desirably, the allogeneic cell line expresses high levels of GM-CSF.

In another aspect, one or more genetically modified GM-CSF expressing allogeneic cell lines can be exposed to an antigen, such that the patient's immune response to the antigen is increased in the presence of GM-CSF, e.g., an allogeneic or bystander cell that has been genetically modified to express GM-CSF. Such exposure may take place ex vivo or in vivo. In one preferred embodiment, the antigen is a peptide comprising an amino acid sequence obtained from COPB2. The COPB2 peptide can be provided by (on) cells that are administered to the subject or may be provided by cells native to the patient. In such cases, the composition can be rendered proliferation-incompetent, typically by irradiation, wherein the allogeneic cells are plated in a tissue culture plate and irradiated at room temperature using a Cs source, as further described herein. An allogeneic cellular immunotherapy composition as described herein may comprise allogeneic cells plus other cells, i.e. a different type of allogeneic cell, an autologous cell, or a bystander cell that may or may not be genetically modified. If genetically modified, the different type of allogeneic cell, autologous cell, or bystander cell may express GM-CSF or another transgene. The ratio of allogeneic cells to other cells in a given administration will vary dependent upon the combination.

Any suitable route of administration can be used to introduce an allogeneic cell line composition into the patient, preferably, the composition is administered intradermally, subcutaneously or intratumorally.

The use of allogeneic cell lines in practicing the methods described herein provides the therapeutic advantage that administration of a genetically modified GM-CSF expressing cell line to a patient with cancer, together with an autologous cancer antigen, paracrine production of GM-CSF results in an effective immune response to a tumor. This obviates the need to culture and transduce autologous tumor cells for each patient.

4.7.3. Bystander Cells

In one further aspect, a universal immunomodulatory genetically modified transgene-expressing bystander cell that expresses at least one transgene can be used in the immunotherapies described herein. The same universal bystander cell line may express more than one transgene, or individual transgenes may be expressed by different universal bystander cell lines. The universal bystander cell line comprises cells which either naturally lack major histocompatibility class I (MHC-I) antigens and major histocompatibility class II (MHC-II) antigens or have been modified so that they lack MHC-I antigens and MHC-II antigens. In one aspect, a universal bystander cell line can be modified by introduction of a vector wherein the vector comprises a nucleic acid sequence encoding a transgene, e.g., a cytokine such as GM-CSF, operably linked to a promoter and expression control sequences necessary for expression thereof. In another aspect, the same universal bystander cell line or a second universal bystander cell line is modified by introduction of a vector comprising a nucleic acid sequence encoding at least one additional transgene operatively linked to a promoter and expression control sequences necessary for expression thereof. The nucleic acid sequence encoding the transgene(s) may be introduced into the same or a different universal bystander cell line using the same or a different vector. The nucleic acid sequence encoding the transgene(s) may or may not further comprise a selectable marker sequence operatively linked to a promoter. Any combination of transgene(s) that stimulate an anti-tumor immune response can be used. The universal bystander cell line preferably grows in defined, i.e., serum-free medium, preferably as a suspension.

An example of a preferred universal bystander cell line is K562 (ATCC CCL-243; Lozzio et al., Blood 45(3): 321-334 (1975); Klein et al., Int. J. Cancer 18: 421-431 (1976)). A detailed description of the generation of human bystander cell lines is described for example in U.S. Pat. No. 6,464,973, expressly incorporated by reference herein.

Desirably, the universal bystander cell line expresses high levels of the transgene, e.g. a cytokine such as GM-CSF.

In the methods, the one or more universal bystander cell lines can be incubated with an autologous cancer antigen, e.g., provided by an autologous tumor cell (which together comprise a universal bystander cell line composition), then the universal bystander cell line composition can be administered to the patient. Any suitable route of administration can be used to introduce a universal bystander cell line composition into the patient. Preferably, the composition is administered intradermally, subcutaneously or intratumorally.

Typically, the autologous cancer antigen can be provided by a cell of the cancer to be treated, i.e., an autologous cancer cell. In such cases, the composition is rendered proliferation-incompetent by irradiation, wherein the bystander cells and cancer cells are plated in a tissue culture plate and irradiated at room temperature using a Cs source, as detailed above.

The ratio of bystander cells to autologous cancer cells in a given administration will vary dependent upon the combination. With respect to GM-CSF-producing bystander cells, the ratio of bystander cells to autologous cancer cells in a given administration should be such that a therapeutically effective level of GM-CSF is produced. In addition to the GM-CSF threshold, the ratio of bystander cells to autologous cancer cells should not be greater than 1:1. Appropriate ratios of bystander cells to tumor cells or tumor antigens can be determined using routine methods known in the art.

The use of bystander cell lines in practicing the methods described herein provides the therapeutic advantage that, through administration of a cytokine-expressing bystander cell line and at least one additional cancer therapeutic agent (expressed by the same or a different cell) to a patient with cancer, together with an autologous cancer antigen, paracrine production of an immunomodulatory cytokine, results in an effective immune response to a tumor. This obviates the need to culture and transduce autologous tumor cells for each patient.

Typically a minimum dose of about 3500 rads is sufficient to inactivate a cell and render it proliferation-incompetent, although doses up to about 30,000 rads are acceptable. In some embodiment, the cells are irradiated at a dose of from about 50 to about 200 rads/min or from about 120 to about 140 rads/min prior to administration to the mammal. Typically, when using irradiation, the levels required are 2,500 rads, 5,000 rads, 10,000 rads, 15,000 rads or 20,000 rads. In one embodiment, a dose of about 10,000 rads is used to inactivate a cell and render it proliferation-incompetent. It is understood that irradiation is but one way to render cells proliferation-incompetent, and that other methods of inactivation which result in cells incapable of multiple rounds of cell division but that retain the ability to express transgenes (e.g. cytokines) are included in the methods provided herein (e.g., treatment with mitomycin C, cycloheximide, and conceptually analogous agents, or incorporation of a suicide gene by the cell).

4.7.4. Cytokines

A “cytokine” or grammatical equivalent, includes, without limitation, those hormones that act locally and do not circulate in the blood, and which, when used in accordance with the methods provided herein, will result in an alteration of an individual's immune response. Also included in the definition of cytokine are adhesion or accessory molecules which result in an alteration of an individual's immune response. Thus, examples of cytokines include, but are not limited to, IL-1 (a or P), IL-2, IL-3, IL-4, IL-5, IL-6, IL-7, IL-8, IL-9, IL-10, IL-11, IL-12, GM-CSF, M-CSF, G-CSF, LIF, LT, TGF-P, γ-IFN, a-EFN, P-IFN, TNF-α, BCGF, CD2, or ICAM. Descriptions of the aforementioned cytokines as well as other applicable immunomodulatory agents may be found in “Cytokines and Cytokine Receptors,” A. S. Hamblin, D. Male (ed.), Oxford University Press, New York, N.Y. (1993)), or the “Guidebook to Cytokines and Their Receptors,” N. A. Nicola (ed.), Oxford University Press, New York, N.Y. (1995)). Where therapeutic use in humans is contemplated, the cytokines will preferably be substantially similar to the human form of the protein or will have been derived from human sequences (i.e., of human origin). In one preferred embodiment, the transgene is a cytokine, such as GM-CSF.

Additionally, cytokines of other mammals with substantial structural homology and/or amino acid sequence identity to the human forms of a given cytokine, will be useful when demonstrated to exhibit similar activity on the human immune system. Similarly, proteins that are substantially analogous to any particular cytokine, but have conservative changes of protein sequence, can also be used. Thus, conservative substitutions in protein sequence may be possible without disturbing the functional abilities of the protein molecule, and thus proteins can be made that function as cytokines in the methods provided herein but have amino acid sequences that differ slightly from currently known sequences. Such conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

Granulocyte-macrophage colony stimulating factor (GM-CSF) is a cytokine produced by fibroblasts, endothelial cells, T cells and macrophages. This cytokine has been shown to induce the growth of hematopoetic cells of granulocyte and macrophage lineages. In addition, it also activates the antigen processing and presenting function of dendritic cells, which are the major antigen presenting cells (APC) of the immune system. Results from animal model experiments have convincingly shown that GM-CSF producing cells are able to induce an immune response against parental, non-transduced cells.

GM-CSF augments the antigen presentation capability of the subclass of dendritic cells (DC) capable of stimulating robust anti-tumor responses (Gasson et al. Blood 1991 Mar. 15; 77(6):1131-45; Mach et al. Cancer Res. 2000 Jun. 15; 60(12):3239-46; reviewed in Mach and Dranoff, Curr Opin Immunol. 2000 October; 12(5):571-5). See, e.g., Boon and Old, Curr Opin Immunol. 1997 Oct. 1; 9(5):681-3). Presentation of tumor antigen epitopes to T cells in the draining lymph nodes is expected to result in systemic immune responses to tumor metastases. Also, irradiated tumor cells expressing GM-CSF have been shown to function as a potent immunotherapy against tumor challenge. Localized high concentrations of certain cytokines, delivered by genetically modified cells, have been found to lead to tumor regression (Abe et al., J. Canc. Res. Clin. Oncol. 121: 587-592 (1995); Gansbacher et al., Cancer Res. 50: 7820-7825 (1990); Formi et al., Cancer and Met. Reviews 7: 289-309 (1988). PCT publication WO200072686 describes tumor cells expressing various cytokines.

In one embodiment, the cellular immunogenic composition comprises a GM-CSF coding sequence operatively linked to regulatory elements for expression in the cells of the immunotherapy. The GM-CSF coding sequence may code for a murine or human GM-CSF and may be in the form of genomic DNA (SEQ ID NO: NO.:84; disclosed as SEQ ID NO: NO.:1 in US Patent Publication NO. 2006/0057127, which is hereby incorporated by reference in its entirety) or cDNA (SEQ ID NO: NO.:85; disclosed as SEQ ID NO: NO.:2 in US Patent Publication NO. 2006/0057127, which is hereby incorporated by reference in its entirety). In the case of cDNA, the coding sequence for GM-CSF does not contain intronic sequences to be spliced out prior to translation. In contrast, for genomic GM-CSF, the coding sequence contains at least one native GM-CSF intron that is spliced out prior to translation. In one embodiment, the GM-CSF coding sequence encodes the amino acid sequence presented as SEQ ID NO.:86 (disclosed as SEQ ID NO.:3 in US Patent Publication NO. 2006/0057127, which is hereby incorporated by reference in its entirety). Other examples of GM-CSF coding sequences are found in Genbank accession numbers: AF373868, AC034228, AC034216, M 10663 and NM000758.

A GM-CSF coding sequence can be a full-length complement that hybridizes to the sequence shown in SEQ ID NO:84 or SEQ ID NO:85 under stringent conditions. The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

It therefore follows that the coding sequence for a cytokine such as GM-CSF, can have at least 80, 85, 87, 89, 90, 91, 92, 93, 94, 95, 96, 97, 98, 99% or more % identity over its entire length to a native GM-CSF coding sequence. For example, a GM-CSF coding sequence can have at least 80, 85, 87, 89, 90, 91, 92, 93, 94, 95, 96, 97, 98, 99% or more sequence identity to a sequence presented as SEQ ID NO: NO:9 or SEQ ID NO: NO:10, when compared and aligned for maximum correspondence, as measured a sequence comparison algorithm (as described above) or by visual inspection. In one embodiment, the given % sequence identity exists over a region of the sequences that is at least about 50 nucleotides in length. In another embodiment, the given % sequence identity exists over a region of at least about 100 nucleotides in length. In another embodiment, the given % sequence identity exists over a region of at least about 200 nucleotides in length. In another embodiment, the given % sequence identity exists over the entire length of the sequence. Preferably, the GM-CSF has authentic GM-CSF activity, e.g., can bind the GM-CSF receptor.

In some embodiments, the amino acid sequence for a cytokine such as GM-CSF has at least 80, 85, 87, 89, 90, 91, 92, 93, 94, 95, 96, 97, 98, 99% or more sequence identity to the sequence presented as SEQ ID NO: NO:86, when compared and aligned for maximum correspondence.

### 5. EXAMPLES

The methods and compositions provided herein are described by reference to the following Examples, which are offered by way of illustration and are not intended to limit the invention in any manner. Standard techniques well known in the art or the techniques specifically described below are utilized. It will be appreciated that the methods and compositions provided herein can be incorporated in the form of a variety of embodiments, only a few of which are disclosed herein. It will be apparent to the artisan that other embodiments exist and do not depart from the spirit of the methods and compositions provided herein. Thus, the described embodiments are illustrative and should not be construed as restrictive.

Exemplary methods for producing recombinant viral vectors useful for making genetically altered tumor cells that express GM-CSF, methods for using the genetically altered tumor cells that express GM-CSF in cancer therapies are extensively described in U.S. Patent Application Publication No. 2006/0057127, incorporated by reference in its entirety, and will not be reproduced below. One such therapy that has been and is being evaluated in clinical trials for treatment of lung cancer is GVAX® therapy.

### 5.1 Example 1

### Identification of Protein Targets of Host Antibody Responses Following Cell-Based Lung Cancer Immunotherapy

This example describes identification of protein targets of host antibody responses following autologous cancer immunotherapy armed with GM-CSF as described above.

The feasibility, safety and efficacy of autologous GM-CSF-secreting cancer immunotherapies have been evaluated in two clinical trials in patients with early and advanced stage non-small cell lung cancer (NSCLC). In the first of these trials, 43 patients were immunized with autologous tumor cells modified with an adenoviral vector to secrete GM-CSF. Intradermal injections of the modified tumor cells were administered 2 weeks for a total of 3-6 immunizations. Three of the 33 advanced-stage patients that were treated in this trial had durable compete tumor responses. In the second clinical trial, 52 patients received at least one immunization, and one had a near complete response in a 2 cm lung mass that remained without progression for >15 months.

Humoral patient immune responses to the autologous non-small cell lung cancer immunotherapy have been evaluated using Serological Analysis of Gene Expression Libraries (SEREX). SEREX allows for the identification of antigens which may be specifically recognized by the patients' immune system following the immunotherapy. From this technique, multiple antibody responses to proteins derived from the immunotherapy have been identified that are specifically induced or augmented following immunization.

5.1.1. Serological Analysis of Gene Expression Libraries (SEREX)

SEREX allows the systematic cloning of tumor antigens recognized by the autoantibody repertoire of cancer patients (Sahin et al. 1995; McNeel et al. 2000; Wang et al. 2005; Dunphy et al. 2005; Qin et al. 2006). cDNA expression libraries were constructed from non-small cell lung cancer (NCIH838 and NCIH1623) and prostate cancer cell lines (PC-3 and LNCaP), packaged into lambda-phage vectors, and expressed recombinantly in E. Coli. Recombinant proteins expressed during the lytic infection of bacteria were then blotted onto nitrocellulose membranes and probed with diluted patient serum for identification of clones reactive with high-titer IgG antibodies.

This procedure was carried out for the 4 patients treated with cell-based lung cancer immunotherapy who showed complete or near complete tumor responses (described above). From the SEREX analysis of these 4 patients, multiple NCIH838/NCIH1623/LNCaP/PC-3 derived cell protein clones reactive to the patient sera post-immunotherapy were identified. Positive antigen hits from the SEREX screen were then screened against pre-immunotherapy serum to determine if the antibody response to these proteins was augmented or induced following the immunotherapy. Table 2, below, provides a compiled list of induced antibody hits (29 proteins) for all 4 patients screened by SEREX. In addition, Table 4 provides positive antigen hits (28 proteins) from the SEREX screen for which a humoral response was detected in at least 2 of the 4 responders. Table 3 provides an additional list of induced antibody hits (34 proteins) identified through SEREX screening. In Table 3, an induction is indicated by an increase in score from pre-immunotherapy serum (“pre”) to post-immunotherapy serum (“post”), whereas a reduction is indicated by a decrease in score from pre to post.

TABLE 2
Induced Antibody Hits

Pre/Post Score

| | | | | | |
|---|---|---|---|---|---|
| Library | LA (Ad) | LB (BAC) | LC (BAC) | LD (SCC) | Summary |

| Gene | PC-3 | LNCaP | H838 | H1623 | serum | plasma | plasma | plasma | Induced | Overlap | Frequency |
|---|---|---|---|---|---|---|---|---|---|---|---|
| COPB2 | | 1 | 1 | | +/++ | +/+ | +/++ | +/++ | 3 | 4 | 2 |
| EPRS | | | 1 | | +/++ | +/++ | +/+ | +/+ | 2 | 4 | 1 |
| DDX41 | | 1 | | | +/++ | ++/+++ | +/+ | +/+ | 2 | 4 | 1 |
| IRAK4 | | | | 3 | +/++ | +++/+++ | ++/+++ | −/− | 2 | 3 | 3 |
| MDH1 | | 1 | | | +/++ | +/++ | −/− | −/− | 2 | 2 | 1 |
| ZRF1 | | 1 | | | +/+ | +/++ | +/+ | +/+ | 1 | 4 | 1 |
| TOP2B | | 1 | 3 | | +/++ | +/+ | +/+ | +/+ | 1 | 4 | 4 |
| AKAP9 | | 1 | | | +/+ | ++/+++ | +/+ | +/+ | 1 | 4 | 1 |
| TMEM33 | | 1 | | | ++/+++ | +++/+++ | ++/++ | +++/+++ | 1 | 4 | 1 |
| SETD1B | | 1 | | | +/++ | +/+ | +/+ | +/+ | 1 | 4 | 1 |
| BDP1 | | 1 | | | +/++ | ++/++ | +/+ | +/+ | 1 | 4 | 1 |
| CEP290 | | | 1 | | +/+ | +/+ | ++/+++ | +/+ | 1 | 4 | 1 |
| AHNAK | 1 | | | | ++/+++ | +/+ | +/+ | −/− | 1 | 3 | 1 |
| PDAP1 | | 2 | | | +/+ | +++/+++ | ++/+++ | −/− | 1 | 3 | 2 |
| ZNF397 | | | | 1 | +/+ | +/++ | +/+ | −/− | 1 | 3 | 1 |
| SMC1A | | 1 | | | +/++ | ++/++ | +/+ | −/− | 1 | 3 | 1 |
| MYO18A | | 1 | | | +/+ | −/− | ++/+++ | +/+ | 1 | 3 | 1 |
| PALLD | | | | 1 | +/+ | ++/+++ | ++/++ | −/− | 1 | 3 | 1 |
| SASS6 | 1 | | | | +/++ | ++/++ | +/+ | −/− | 1 | 3 | 1 |
| PSAT1 | 1 | | | | +/++ | +/+ | −/− | −/− | 1 | 2 | 1 |
| VCP | | 4 | 1 | | ++/+++ | −/− | +/+ | −/− | 1 | 2 | 5 |
| BAZ2B | | | 5 | | +/++ | −/− | +/+ | −/− | 1 | 2 | 5 |
| GARNL1 | | | | 1 | +/+ | −/+ | −/− | −/− | 1 | 2 | 1 |
| Hypothetical | | | 2 | | ++/+++ | −/− | −/− | ++/++ | 1 | 2 | 2 |
| DKFZP686A01247 | | | | | | | | | | | |
| NAP1L1 | 1 | | 1 | | +/++ | −/− | −/− | −/− | 1 | 1 | 2 |
| PNMA1 | 1 | | | | ++/+++ | −/− | −/− | −/− | 1 | 1 | 1 |
| HNRPA1 | | | 1 | | +/++ | +/+ | −/− | −/− | 1 | 2 | 1 |
| PTPRF | 1 | | | | −/− | ++/+++ | −/− | −/− | 1 | 1 | 1 |
| POH1 | | | 1 | | +/++ | −/− | −/− | −/− | 1 | 1 | 1 |

TABLE 3
Induced Antibody Hits

| | | Patient | | Patient | | Patient | | Patient | | Patient | |
|---|---|---|---|---|---|---|---|---|---|---|---|
| | | A | | B | | C | | D | | E | |
| Gene | Genbank # | Pre | Post | Pre | Post | Pre | Post | Pre | Post | Pre | Post |
| RSN | NM_198240 | 2 | 2 | 1 | 2 | 3 | 3 | 1 | 2 | 1 | 2 |
| DARS2 | NM_018122 | 1 | 2 | 1 | 2 | 2 | 2 | 1 | 1 | 1 | 2 |
| KTN1 | NM_182926 | 2 | 2 | 2 | 2 | 2 | 3 | 2 | 3 | 2 | 1 |
| CENPF | NM_016343 | 3 | 2 | 1 | 2 | 1 | 1 | 1 | 1 | 1 | 2 |
| IRAK4 | NM_016123 | 1 | 2 | 3 | 2 | 2 | 3 | 1 | 1 | 0 | 0 |
| REST | NM_005612 | 1 | 1 | 1 | 3 | 1 | 1 | 1 | 1 | 1 | 1 |
| AKAP9 | NM_147171 | 1 | 1 | 1 | 2 | 2 | 2 | 1 | 1 | 2 | 2 |
| BAZ2B | NM_013450 | 2 | 3 | 1 | 1 | 3 | 3 | 1 | 1 | 1 | 1 |
| BRAP | NM_006768 | 2 | 2 | 2 | 2 | 1 | 1 | 1 | 1 | 1 | 2 |
| COPB2 | NM_004766 | 1 | 1 | 2 | 2 | 1 | 1 | 1 | 1 | 1 | 2 |
| UBTF | NM_014233 | 1 | 2 | 1 | 1 | 1 | 1 | 1 | 1 | 1 | 1 |
| TPR | NM_003292 | 1 | 1 | 1 | 1 | 2 | 3 | 1 | 1 | 1 | 1 |
| PSMD7 | NM_002811 | 1 | 2 | 0 | 0 | 1 | 1 | 1 | 1 | 2 | 2 |
| NRBF2 | NM_030759 | 0 | 0 | 0 | 0 | 1 | 1 | 3 | 2 | 1 | 2 |
| SUHW4 | NM_017661 | 2 | 3 | 1 | 1 | 1 | 1 | 0 | 0 | 0 | 0 |
| BRWD1 | NM_033656 | 2 | 1 | 0 | 0 | 2 | 3 | 0 | 0 | 0 | 0 |
| ROCK2 | NM_004850 | 0 | 0 | 3 | 1 | 1 | 1 | 0 | 0 | 1 | 2 |
| MAML3 | NM_018717 | 1 | 1 | 0 | 0 | 2 | 2 | 1 | 2 | 0 | 0 |
| $$DCCAG8 | NM_006642 | 1 | 3 | 1 | 1 | 0 | 0 | 0 | 0 | 0 | 0 |
| NAP1L1 | NM_004537 | 1 | 3 | 1 | 1 | 0 | 0 | 0 | 0 | 0 | 0 |
| FAM50A | NM_004699 | 1 | 2 | 0 | 0 | 0 | 0 | 1 | 1 | 0 | 0 |
| SLMAP | NM_007159 | 1 | 2 | 0 | 0 | 1 | 1 | 0 | 0 | 0 | 0 |
| FAF1 | NM_131917 | 0 | 0 | 1 | 2 | 0 | 0 | 0 | 0 | 1 | 1 |
| ETNK1 | NM_001039481 | 1 | 1 | 0 | 0 | 1 | 2 | 0 | 0 | 0 | 0 |
| ICA1 | NM_004968 | 1 | 3 | 0 | 0 | 0 | 0 | 0 | 0 | 0 | 0 |
| ARL6IP5 | NM_006407 | 2 | 3 | 0 | 0 | 0 | 0 | 0 | 0 | 0 | 0 |
| POLR1B | NM_019014 | 2 | 3 | 0 | 0 | 0 | 0 | 0 | 0 | 0 | 0 |
| STK39 | NM_013233 | 1 | 2 | 0 | 0 | 0 | 0 | 0 | 0 | 0 | 0 |
| UHMK1 | NM_175866 | 0 | 0 | 2 | 3 | 0 | 0 | 0 | 0 | 0 | 0 |
| CHMP4C | NM_152284 | 0 | 0 | 2 | 3 | 0 | 0 | 0 | 0 | 0 | 0 |
| ZNF397 | NM_032347 | 0 | 0 | 2 | 3 | 0 | 0 | 0 | 0 | 0 | 0 |
| TRMT5 | NM_020810 | 0 | 0 | 0 | 0 | 1 | 2 | 0 | 0 | 0 | 0 |
| RPIA | NM_144563 | 0 | 0 | 0 | 0 | 1 | 2 | 0 | 0 | 0 | 0 |
| SPATA5 | NM_145207 | 0 | 0 | 0 | 0 | 2 | 3 | 0 | 0 | 0 | 0 |

TABLE 4
Positive antigen hits detected in at least 2 of 4 responders

| | | | | Patient |
|---|---|---|---|---|
| Name | Genbank # | SEQ ID NO | Identity | overlap |
| AKAP9 | NM_147171, | SEQ ID | A kinase (PRKA) anchor | 2 |
| | NT_007933 | NO: 23; | protein (yotiao) 9 | |
| | | SEQ ID | (AKAP9), transcript | |
| | | NO: 112 | variant 1 | |
| ATRX | NM_138270, | SEQ ID | alpha | 2 |
| | NM_000489 | NO: 113; | thalassemia/mental | |
| | | SEQ ID | retardation syndrome X- | |
| | | NO: 114 | linked (RAD54 homolog, | |
| | | | S. cerevisiae) (ATRX) | |
| BAZ2B | NM_013450 | SEQ ID | bromodomain adjacent to | 2 |
| | | NO: 59 | zinc finger domain 2B | |
| | | | (BAZ2B) | |
| BRAP | NM_006768 | SEQ ID | BRCA1 associated | 2 |
| | | NO: 102 | protein (BRAP) | |
| CENPF | NM_016343 | SEQ ID | centromere protein F, | 3 |
| | | NO: 87 | 350/400ka (mitosin) | |
| | | | (CENPF) | |
| COPB2 | NM_004766 | SEQ ID | coatomer protein | 3 |
| | | NO: 2 | complex, subunit beta 2 | |
| | | | (beta prime) (COPB2) | |
| CSF2 | NM_000758 | | colony stimulating factor | 2 |
| | | | 2 (granulocyte- | |
| | | | macrophage) (CSF2) | |
| EPRS | NM_004446 | SEQ ID | glutamyl-prolyl-tRNA | 2 |
| | | NO: 4 | synthetase (EPRS) | |
| GCC2 | NM_181453 | SEQ ID | GRIP and coiled-coil | 3 |
| | | NO: 115 | domain containing 2 | |
| | | | (GCC2), transcript | |
| | | | variant 1 | |
| GOLGA4 | NM_002078 | SEQ ID | golgi autoantigen, golgin | 4 |
| | | NO: 116 | subfamily a, 4 (GOLGA4) | |
| HSP90AA1 | NM_005348 | SEQ ID | heat shock protein | 2 |
| | | NO: 117 | 90 kDa alpha (cytosolic), | |
| | | | class A member 1 | |
| | | | (HSP90AA1), transcript | |
| | | | variant 2 | |
| IRAK4 | NM_016123 | SEQ ID | interleukin-1 receptor- | 2 |
| | | NO: 8 | associated kinase 4 | |
| | | | (IRAK4) | |
| KTN1 | NM_182926 | SEQ ID | kinectin 1 (kinesin | 3 |
| | | NO: 86 | receptor) (KTN1) | |
| LRRFIP1 | NM_004735 | SEQ ID | leucine rich repeat (in | 2 |
| | | NO: 79 | FLII) interacting protein 1 | |
| | | | (LRRFIP1) | |
| MCM3 | NM_002388 | SEQ ID | MCM3 minichromosome | 2 |
| | | NO: 118 | maintenance deficient 3 | |
| | | | (S. cerevisiae) (MCM3) | |
| MPHOSPH1 | NM_016195 | SEQ ID | M-phase phosphoprotein | 2 |
| | | NO: 119 | 1 (MPHOSPH1) | |
| PARP4 | NM_006437 | SEQ ID | poly (ADP-ribose) | 2 |
| | | NO: 120 | polymerase family, | |
| | | | member 4 (PARP4) | |
| PHF3 | NM_015153 | SEQ ID | PHD finger protein 3 | 2 |
| | | NO: 121 | (PHF3) | |
| PPP1R9A | NM_017650 | SEQ ID | protein phosphatase 1, | 2 |
| | | NO: 122 | regulatory (inhibitor) | |
| | | | subunit 9A (PPP1R9A) | |
| RIOK1 | NM_031480 | SEQ ID | RIO kinase 1 (yeast) | 2 |
| | | NO: 123 | (RIOK1), transcript | |
| | | | variant 1 | |
| ROCK1 | NM_005406 | SEQ ID | Rho-associated, coiled- | 3 |
| | | NO: 124 | coil containing protein | |
| | | | kinase 1 (ROCK1) | |
| SMC2 | NM_006444 | SEQ ID | structural maintenance of | 2 |
| | | NO: 125 | chromosomes 2 (SMC2) | |
| TMF1 | NM_007114 | SEQ ID | TATA element | 3 |
| | | NO: 126 | modulatory factor 1 | |
| | | | (TMF1) | |
| TOP2B | NM_001068 | SEQ ID | topoisomerase (DNA) II | 2 |
| | | NO: 16 | beta 180 kDa (TOP2B) | |
| TWISTNB | NM_001002926 | SEQ ID | TWIST neighbor | 2 |
| | | NO: 127 | (TWISTNB) | |
| UBTF | NM_014233 | SEQ ID | upstream binding | 2 |
| | | NO: 89 | transcription factor, RNA | |
| | | | polymerase I (UBTF) | |
| ZNF638 | NM_014497 | SEQ ID | zinc finger protein 638 | 3 |
| | | NO: 128 | (ZNF638), transcript | |
| | | | variant 1 | |
| ZRF1 | NM_014377 | SEQ ID | zuotin related factor 1 | 3 |
| | | NO: 14 | (ZRF1) | |

5.1.2. Cloning and Characterization of Antigens

Full length genes are cloned into a mammalian based expression system (e.g., a lentiviral expression plasmid) and a FLAG-tag is added at the C-terminal end to aid with detection and purification. Antibody responses to high frequency hits of proteins are determined from all trials available and the induction of antibody response is examined in correlation to survival. These responses are used for a number of applications including use as surrogate markers of immunotherapy treatment, correlation with patient survival data to provide an efficacy signature, clinical trial monitoring (biomarkers) and assay development of cell characterization marker for lot release (product characterization, comparability markers).

Following identification of proteins correlated with an antibody response in serum, antigen targets may be further characterized for the presence of a cellular immune response (T-cells) in cases for which high quality peripheral blood mononuclear cells (PBMCs) harvested from patients administered cell-based lung cancer immunotherapy are available.

5.1.2.1 Detecting Activation of Cytotoxic T Lymphocytes in IFN-γ Assays

This example provides an exemplary method for detecting activation of cytotoxic T lymphocytes (CTLs) by monitoring IFN-γ expression by the CTLs in response to exposure to an appropriate antigen, e.g., a COPB2 peptide presented on an MHC I receptor.

First, peripheral blood monocytic cells (PBMCs) are isolated from a subject to be assessed for cellular immune response against a COPB2 peptide and CD8+ cells are isolated by fluorescence activated cell sorting (FACS). The CD8+ cells are then incubated with, e.g., T2 cells loaded with the COPB2 peptide to be assessed, produced as described above, and in the presence of suitable cytokines for expanding the CTL population.

IFN-γ release by the CTLs is measured using an IFN-γ ELISA kit (PBL-Biomedical Laboratory, Piscataway, N.J.). Briefly, purified IFN-γ as standards or culture supernates from the CTL-T2 co-culture are transferred into wells of a 96-well plate pre-coated with a monoclonal anti-human IFN-γ capture antibody and incubated for 1 h in a closed chamber at 24° C. After washing the plate with PBS/0.05% Tween 20, biotin anti-human IFN-γ antibody is added to the wells and incubated for 1 h at 24° C. The wells are washed and then developed by incubation with streptavidin horseradish peroxidase conjugate and TMB substrate solution. Stop solution is added to each well and the absorbance is determined at 450 nm with a SpectraMAX Plus plate reader (Stratagene, La Jolla, Calif.). The amount of cytokine present in the CTL culture supernatants is calculated based on the IFN-γ standard curve.

5.1.2.2 Detecting Activation of Cytotoxic T Lymphocytes in Proliferation Assays

This example provides an exemplary method for detecting activation of cytotoxic T lymphocytes (CTLs) by CTL proliferation in response to exposure to an appropriate antigen, e.g., a COPB2 peptide presented on an MHC I receptor.

First, peripheral blood monocytic cells (PBMCs) are isolated from a subject to be assessed for cellular immune response against a COPB2 peptide and CD8+ cells are isolated by fluorescence activated cell sorting (FACS). The CD8+ cells are then incubated with, e.g., T2 cells loaded with the COPB2 peptide to be assessed, produced as described above.

Next, the samples are incubated for 12 hours, then 20 μl of 3H-thymidine is added to each well and the sample incubated for an additional 12 hours. Cells are harvested and the plate is read in a beta counter to determine the amount of unincorporated 3H-thymidine.

5.1.2.3 Detecting Activation of Cytotoxic T Lymphocytes in Effector Assays

This example provides an exemplary method for detecting activation of cytotoxic T lymphocytes (CTLs) by monitoring lysis of cells displaying an appropriate antigen, e.g., a COPB2 peptide presented on an MHC I receptor.

The cytotoxic activity of the CTLs is measured in a standard ⁵¹Cr-release assay. Effector cells (CTLs) are seeded with ⁵¹Cr-labeled target cells (5×10³ cells/well) at various effector:target cell ratios in 96-well U-bottom microtiter plates. Plates are incubated for 4 h at 37° C., 5% CO₂. The ⁵¹Cr-release is measured in 100 μl supernatant using a Beckman LS6500 liquid scintillation counter (Beckman Coulter, Brea, Calif.). The percent specific cell lysis is calculated as [(experimental release−spontaneous release)/(maximum release−spontaneous release)]. Maximum release is obtained from detergent-released target cell counts and spontaneous release from target cell counts in the absence of effector cells.

### REFERENCES

Casiano C A, Mediavilla-Varela M, Tan E M. Tumor-associated antigen arrays for the serological diagnosis of cancer. Mol Cell Proteomics. 2006 October; 5(10):1745-59. Epub 2006 May 29. Review. PMID: 16733262.
Bradford T J, Wang X, Chinnaiyan A M. Cancer immunomics: using autoantibody signatures in the early detection of prostate cancer. Urol Oncol. 2006 May-June; 24(3):237-42. PMID: 16678056.
Qin S, Qiu W, Ehrlich J R, Ferdinand A S, Richie J P, O'leary M P, Lee M L, Liu B C. Development of a “reverse capture” autoantibody microarray for studies of antigen-autoantibody profiling. Proteomics. 2006 Apr. 5;
Wang X, Yu J, Sreekumar A, Varambally S, Shen R, Giacherio D, Mehra R, Montie J E, Pienta K J, Sanda M G, Kantoff P W, Rubin M A, Wei J T, Ghosh D, Chinnaiyan A M. Autoantibody signatures in prostate cancer. N Engl J. Med. 2005 Sep. 22; 353(12):1224-35.
Dunphy E J, McNeel D G. Antigen-specific IgG elicited in subjects with prostate cancer treated with flt3 ligand. J. Immunother. 2005 May-June; 28(3):268-75.
McNeel D G, Nguyen L D, Storer B E, Vessella R, Lange P H, Disis M L. Antibody immunity to prostate cancer associated antigens can be detected in the serum of patients with prostate cancer. J. Urol. 2000 November; 164(5):1825-9.
Sahin U, Tureci O, Schmitt H, Cochlovius B, Johannes T, Schmits R, Stenner F, Luo G, Schobert I, Pfreundschuh M. Human neoplasms elicit multiple specific immune responses in the autologous host. Proc Natl Acad Sci USA. 1995 Dec. 5; 92(25):11810-3.
Varambally S, Yu J, Laxman B, Rhodes D R, Mehra R, Tomlins S A, Shah R B, Chandran U, Monzon F A, Becich M J, Wei J T, Pienta K J, Ghosh D, Rubin M A, Chinnaiyan A M. Integrative genomic and proteomic analysis of prostate cancer reveals signatures of metastatic progression. Cancer Cell. 2005 November; 8(5):393-406.

While many specific examples have been provided, the above description is intended to illustrate rather than limit the invention. Many variations of the invention will become apparent to those skilled in the art upon review of this specification. The scope of the invention should, therefore, be determined not with reference to the above description, but instead should be determined with reference to the appended claims along with their full scope of equivalents.

All sequences referenced by accession number, publications, and patent documents cited in this application are incorporated by reference in their entirety for all purposes to the same extent as if each individual publication or patent document were so individually denoted. Citation of these documents is not an admission that any particular reference is “prior art” to this invention.

What is claimed is:

1. A method for identifying whether a subject is afflicted with lung cancer, comprising detecting an immune response against an antigen identified in Table 2, 3 or 4, wherein detection of the immune response indicates that the subject is afflicted with lung cancer.

2. The method of claim 1, wherein the lung cancer is non-small cell lung cancer.

3. The method of claim 1, wherein the subject is a mammal.

4. The method of claim 1, wherein the subject is a human.

5. The method of claim 1, wherein the immune response is a humoral immune response.

6. The method of claim 1, wherein the immune response is a cellular immune response.

7. The method of claim 1, wherein an immune response is detected against 1, 2, 3, 4, 5, 6, 7, 8, 9, 10, 11, 12, 13, 14, or 15 of the antigens in Table 2, 3 or 4.

8. A method for determining whether a subject is likely to respond to lung cancer therapy with a composition comprising cancer cells that have been rendered proliferation-incompetent and have been genetically engineered to express GM-CSF, comprising detecting an immune response against an antigen listed in Table 2, 3 or 4, wherein detecting the immune response indicates that the subject is likely to respond to said lung cancer therapy.

9. The method of claim 8, wherein the lung cancer therapy is for the treatment of non-small cell lung cancer.

10. The method of claim 8, wherein the subject is a mammal.

11. The method of claim 8, wherein the subject is a human.

12. The method of claim 8, wherein the cancer cells are autologous.

13. The method of claim 8, wherein the cancer cells are allogeneic.

14. The method of claim 8, wherein an immune response is detected against one, two, three, four, five, six, seven, eight, nine, ten, eleven, twelve, thirteen, fourteen, fifteen, or more of the antigens listed in Table 2, 3 or 4.

15. The method of claim 8, wherein responsiveness to the cancer therapy is measured by decreased serum concentrations of tumor specific markers, increased overall survival time, increased progression-free survival, decreased tumor size, decreased metastasis marker response, increased impact on minimal residual disease, increased induction of antibody response to the cancer cells that have been rendered proliferation-incompetent, increased induction of delayed-type-hypersensitivity (DTH) response to injections of autologous tumor, increased induction of T cell response to autologous tumor or candidate tumor-associated antigens, increased impact on circulating T cell and dendritic cell numbers, phenotype, and function, cytokine response, reduced metastasis as measured by bone scan/MRI, increased time to progression, decreased serum concentrations of ICTP, decreased concentrations of serum C-reactive protein or decreased numbers of circulating tumor cells.

16. The method of claim 8, wherein responsiveness to the cancer therapy is measured by decreased serum concentrations of tumor specific markers.

17. The method of claim 8, wherein responsiveness to the cancer therapy is measured by increased overall survival time.

18. The method of claim 8, wherein responsiveness to the cancer therapy is measured by increased progression-free survival.

19. The method of claim 8, wherein responsiveness to the cancer therapy is measured by decreased tumor size.

20. The method of claim 8, wherein responsiveness to the cancer therapy is measured by decreased metastasis marker response.

21. The method of claim 8, wherein responsiveness to the cancer therapy is measured by increased impact on minimal residual disease.

22. The method of claim 8, wherein responsiveness to the cancer therapy is measured by increased induction of antibody response to the cancer cells that have been rendered proliferation-incompetent.

23. The method of claim 8, wherein responsiveness to the cancer therapy is measured by increased induction of delayed-type-hypersensitivity (DTH) response to injections of autologous tumor.

24. The method of claim 8, wherein responsiveness to the cancer therapy is measured by increased induction of T cell response to autologous tumor or candidate tumor-associated antigens.

25. The method of claim 8, wherein responsiveness to the cancer therapy is measured by increased impact on circulating T cell and dendritic cell numbers, phenotype, and function.

26. The method of claim 8, wherein responsiveness to the cancer therapy is measured by reduced metastasis as measured by bone scan/MRI.

27. The method of claim 8, wherein responsiveness to the cancer therapy is measured by increased time to progression.

28. The method of claim 8, wherein responsiveness to the cancer therapy is measured by decreased serum concentrations of ICTP.

29. The method of claim 8, wherein responsiveness to the cancer therapy is measured by decreased concentrations of serum C-reactive protein.

30. The method of claim 8, wherein the immune response is a humoral immune response.

31. The method of claim 8, wherein the immune response is a cellular immune response.

32. A computer-implemented method for determining whether a subject is likely to respond to lung cancer therapy with a composition comprising cancer cells that have been rendered proliferation-incompetent and have been genetically engineered to express GM-CSF, comprising inputting into a computer memory data indicating whether an immune response against an antigen listed in Table 2, 3 or 4 is detected, inputting into the computer memory a correlation between an immune response against an antigen listed in Table 2, 3 or 4 and a likelihood of responding to said therapy, and determining whether the subject is likely to respond to said therapy.

33.-55. (canceled)

56. Computer-readable media embedded with computer executable instructions for performing the method of claim 32.

57. A computer system configured to perform the method of claim 32.

58. A method for determining whether a subject is responding to lung cancer therapy with a composition comprising cancer cells that have been rendered proliferation-incompetent and have been genetically engineered to express GM-CSF, comprising administering an effective amount of a composition comprising cancer cells that have been rendered proliferation-incompetent and have been genetically engineered to express GM-CSF, and detecting an immune response against an antigen listed in Table 2, 3 or 4, wherein detecting the immune response indicates that the subject is responding to said therapy.

59.-81. (canceled)

82. A computer-implemented method for determining whether a subject is responding to lung cancer therapy with a composition comprising cancer cells that have been rendered proliferation-incompetent and have been genetically engineered to express GM-CSF, comprising administering an effective amount of a composition comprising cancer cells that have been rendered proliferation-incompetent and have been genetically engineered to express GM-CSF, inputting into a computer memory data indicating whether an immune response against an antigen listed in Table 2, 3 or 4 is detected, inputting into the computer memory a correlation between an immune response against an antigen listed in Table 2, 3 or 4 and responsiveness to said therapy, and determining whether the subject is responding to said therapy.

83.-105. (canceled)

106. Computer-readable media embedded with computer executable instructions for performing the method of claim 82.

107. A computer system configured to perform the method of claim 82.

108. A method for determining whether a subject is responding to lung cancer therapy with a composition comprising cancer cells that have been rendered proliferation-incompetent and have been genetically engineered to express GM-CSF, comprising detecting an immune response against an antigen listed in Table 2, 3 or 4 at a first time, administering an effective amount of a composition comprising cancer cells that have been rendered proliferation-incompetent and have been genetically engineered to express GM-CSF, and detecting an immune response against the antigen listed in Table 2, 3 or 4 at a later second time, wherein an increase in the immune response detected at the later second time relative to the earlier first time indicates that the subject is responding to said therapy.

109.-131. (canceled)

132. A computer-implemented method for determining whether a subject is responding to lung cancer therapy with a composition comprising cancer cells that have been rendered proliferation-incompetent and have been genetically engineered to express GM-CSF, comprising administering an effective amount of a composition comprising cancer cells that have been rendered proliferation-incompetent and have been genetically engineered to express GM-CSF, inputting into a computer memory data indicating whether an immune response against an antigen listed in Table 2, 3 or 4 is detected at a first time prior to said step of administering and at a later second time subsequent to said step of administering, inputting into the computer memory a correlation between an increase in the immune response against the antigen listed in Table 2, 3 or 4 at said later second time relative to said earlier first time and responsiveness to said therapy, and determining whether the subject is responding to said therapy.

133.-155. (canceled)

156. Computer-readable media embedded with computer executable instructions for performing the method of claim 132.

157. (canceled)

158. (canceled)
